(19) United States
(12) Patent Application Publication
(10) Pub. No.: US 2025/0161276 A1
(43) Pub. Date: May 22, 2025

(54) DRUG COMBINATIONS AND METHODS OF TREATING OVARIAN CANCER

(71) Applicant: Pattern Computer, Inc., Friday Harbor, WA (US)

(72) Inventors: Nidhi SINGH, Friday Harbor, WA (US); Alejandro WOLF-YADLIN, Friday Harbor, WA (US)

(21) Appl. No.: 18/838,982

(22) PCT Filed: Feb. 17, 2023
(86) PCT No.: PCT/US2023/062827

§ 371 Date: Aug. 15, 2024

Related U.S. Application Data
(60) Provisional application No. 63/311,719, filed on Feb. 18, 2022.

(51) Int. Cl. A61K 31/4184; A61K 31/4523; A61K 31/506; A61K 31/517; A61K 31/519; A61K 31/5375; A61K 31/5377

(52) U.S. Cl. CPC A61K 31/4184; A61K 31/4523; A61K 31/506; A61K 31/517; A61K 31/519; A61K 31/5375; A61K 31/5377

(57) ABSTRACT

The present disclosure provides therapeutic combinations effective in the treatment of cancer. The present disclosure also provides methods of treatment, including administration of the combinations, and uses of the combinations, e.g., for the treatment of cancer.

### CROSS-REFERENCE TO RELATED APPLICATIONS

This application claims the benefit under 35 U.S.C. § 119 (e) of U.S. Provisional Patent Application No. 63/311,719, filed Feb. 18, 2022, the entire disclosure of which is hereby incorporated herein by reference.

### BACKGROUND

Cancer is a general term used to describe diseases in which abnormal cells divide without control. Cancer cells can invade adjacent tissues and can spread through the bloodstream and lymphatic system to other parts of the body. There are different types of cancers such as the bladder cancer, breast cancer, colon cancer, rectal cancer, head and neck cancer, endometrial cancer, kidney (renal cell) cancer, leukemia, small cell lung cancer, non-small cell lung cancer, ovarian cancer, pancreatic cancer, prostate cancer, thyroid cancer, skin cancer, Non-Hodgkin's Lymphoma and melanoma. Ovarian cancer often goes unnoticed in the early-stages and is only detected once it has spread within the pelvis and abdomen. At this late stage, ovarian cancer is considerably more difficult to treat. High-grade serous carcinoma is the most malignant form of ovarian cancer and accounts for up to 70% of all ovarian cancer cases. High-grade serous ovarian cancers originate in the fallopian tubes and spread through the abdomen early during the course of the disease. Accordingly, by the time these cancers become symptomatic, they are usually high stage tumors with resultingly poor outcomes.

Thus, there is a need to develop new treatments for cancers, including but not limited to ovarian cancer, and high-grade serous ovarian cancer in particular.

### SUMMARY

The present disclosure provides, inter alia, a combination comprising a therapeutically effective amount of binimetinib and a therapeutically effective amount of copanlisib. In some embodiments, the binimetinib and copanlisib are provided in synergistically effective amounts. In some embodiments, the molar ratio of binimetinib to copanlisib is between about 23:1 to about 43:1. In some embodiments, the molar ratio of binimetinib to copanlisib is between about 28:1 to about 38:1. In some embodiments, the molar ratio of binimetinib to copanlisib is about 33:1. In some embodiments, the molar ratio of binimetinib to copanlisib is between about 1:4 to about 1:10. In some embodiments, the molar ratio of binimetinib to copanlisib is between about 1:6 to about 1:8. In some embodiments, the molar ratio of binimetinib to copanlisib is about 1:7.

The present disclosure further provides a pharmaceutical composition comprising a combination of binimetinib and copanlisib, as described herein, and a pharmaceutically acceptable carrier.

The present disclosure further provides a method of treating cancer in a subject in need thereof, comprising administering to the subject a therapeutically effective amount of a combination of binimetinib and copanlisib, as described herein. In some embodiments, the cancer is ovarian cancer. In some embodiments, the ovarian cancer is selected from high-grade serous ovarian carcinoma, clear cell ovarian carcinoma, endometrioid ovarian carcinoma, mucinous ovarian carcinoma, and low-grade serous ovarian carcinoma. In some embodiments, the ovarian cancer is high-grade serous ovarian cancer. In some embodiments, the ovarian cancer is high-grade serous ovarian adenocarcinoma. In some embodiments, the ovarian cancer is characterized by mutation of the TP53 gene. In some embodiments, the administration of binimetinib and copanlisib is simultaneous. In some embodiments, the administration of binimetinib and copanlisib is sequential.

The present disclosure further provides a combination comprising a therapeutically effective amount of cobimetinib and a therapeutically effective amount of buparlisib. In some embodiments, the cobimetinib and buparlisib are provided in synergistically effective amounts. In some embodiments, the molar ratio of cobimetinib to buparlisib is between about 23:1 to about 43:1. In some embodiments, the molar ratio of cobimetinib to buparlisib is between about 28:1 to about 38:1. In some embodiments, the molar ratio of cobimetinib to buparlisib is about 33:1. In some embodiments, the molar ratio of cobimetinib to buparlisib is between about 5:1 to about 15:1. In some embodiments, the molar ratio of cobimetinib to buparlisib is between about 8:1 to about 12:1. In some embodiments, the molar ratio of cobimetinib to buparlisib is about 10:1.

The present disclosure further provides a pharmaceutical composition comprising a combination of cobimetinib and buparlisib, as described herein, and a pharmaceutically acceptable carrier.

The present disclosure further provides a method of treating cancer in a subject in need thereof, comprising administering to the subject a therapeutically effective amount of a combination of cobimetinib and buparlisib, as described herein. In some embodiments, the cancer is ovarian cancer. In some embodiments, the ovarian cancer is selected from high-grade serous ovarian carcinoma, clear cell ovarian carcinoma, endometrioid ovarian carcinoma, mucinous ovarian carcinoma, and low-grade serous ovarian carcinoma. In some embodiments, the ovarian cancer is high-grade serous ovarian cancer. In some embodiments, the ovarian cancer is high-grade serous ovarian adenocarcinoma. In some embodiments, the ovarian cancer is characterized by mutation of the TP53 gene. In some embodiments, the administration of cobimetinib and buparlisib is simultaneous. In some embodiments, the administration of cobimetinib and buparlisib is sequential.

The present disclosure further provides a combination comprising a therapeutically effective amount of carfilzomib and a therapeutically effective amount of pazopanib. In some embodiments, the carfilzomib and pazopanib are provided in synergistically effective amounts. In some embodiments, the molar ratio of carfilzomib to pazopanib is between about 1:1 to about 6:1. In some embodiments, the molar ratio of carfilzomib to pazopanib is between about 2:1 to about 5:1. In some embodiments, the molar ratio of carfilzomib to pazopanib is about 3:1. In some embodiments, the molar ratio of carfilzomib to pazopanib is about 4:1.

The present disclosure further provides a pharmaceutical composition comprising a combination of carfilzomib and pazopanib, as described herein, and a pharmaceutically acceptable carrier.

The present disclosure further provides a method of treating cancer in a subject in need thereof, comprising administering to the subject a therapeutically effective amount of a combination of carfilzomib and pazopanib, as described herein. In some embodiments, the cancer is ovarian cancer. In some embodiments, the ovarian cancer is selected from high-grade serous ovarian carcinoma, clear cell ovarian carcinoma, endometrioid ovarian carcinoma, mucinous ovarian carcinoma, and low-grade serous ovarian carcinoma. In some embodiments, the ovarian cancer is high-grade serous ovarian cancer. In some embodiments, the ovarian cancer is high-grade serous ovarian adenocarcinoma. In some embodiments, the ovarian cancer is characterized by mutation of the TP53 gene. In some embodiments, the administration of carfilzomib and pazopanib is simultaneous. In some embodiments, the administration of carfilzomib and pazopanib is sequential.

The present disclosure further provides a combination comprising a therapeutically effective amount of copanlisib and a therapeutically effective amount of dasatinib. In some embodiments, the copanlisib and dasatinib are provided in synergistically effective amounts. In some embodiments, the molar ratio of copanlisib to dasatinib is between about 23:1 to about 43:1. In some embodiments, the molar ratio of copanlisib to dasatinib is between about 28:1 to about 38:1. In some embodiments, the molar ratio of copanlisib to dasatinib is about 33:1.

The present disclosure further provides a pharmaceutical composition comprising a combination of copanlisib and dasatinib, as described herein, and a pharmaceutically acceptable carrier.

The present disclosure further provides a method of treating cancer in a subject in need thereof, comprising administering to the subject a therapeutically effective amount of a combination of copanlisib and dasatinib, as described herein. In some embodiments, the cancer is ovarian cancer. In some embodiments, the ovarian cancer is selected from high-grade serous ovarian carcinoma, clear cell ovarian carcinoma, endometrioid ovarian carcinoma, mucinous ovarian carcinoma, and low-grade serous ovarian carcinoma. In some embodiments, the ovarian cancer is high-grade serous ovarian cancer. In some embodiments, the ovarian cancer is high-grade serous ovarian adenocarcinoma. In some embodiments, the ovarian cancer is characterized by mutation of the TP53 gene. In some embodiments, the administration of copanlisib and dasatinib is simultaneous. In some embodiments, the administration of copanlisib and dasatinib is sequential.

The present disclosure further provides a combination comprising a therapeutically effective amount of carfilzomib and a therapeutically effective amount of vandetanib. In some embodiments, the carfilzomib and vandetanib are provided in synergistically effective amounts. In some embodiments, the molar ratio of carfilzomib to vandetanib is between about 23:1 to about 43:1. In some embodiments, the molar ratio of carfilzomib to vandetanib is between about 28:1 to about 38:1. In some embodiments, the molar ratio of carfilzomib to vandetanib is about 33:1. In some embodiments, the molar ratio of carfilzomib to vandetanib is between about 75:1 to about 175:1. In some embodiments, the molar ratio of carfilzomib to vandetanib is between about 100:1 to about 150:1. In some embodiments, the molar ratio of carfilzomib to vandetanib is about 125:1. In some embodiments, the molar ratio of carfilzomib to vandetanib is between about 4:1 to about 8:1. In some embodiments, the molar ratio of carfilzomib to vandetanib is between about 5:1 to about 7:1. In some embodiments, the molar ratio of carfilzomib to vandetanib is about 6:1.

The present disclosure further provides a pharmaceutical composition comprising a combination of carfilzomib and vandetanib, as described herein, and a pharmaceutically acceptable carrier.

The present disclosure further provides a method of treating cancer in a subject in need thereof, comprising administering to the subject a therapeutically effective amount of a combination of carfilzomib and vandetanib, as described herein. In some embodiments, the cancer is ovarian cancer. In some embodiments, the ovarian cancer is selected from high-grade serous ovarian carcinoma, clear cell ovarian carcinoma, endometrioid ovarian carcinoma, mucinous ovarian carcinoma, and low-grade serous ovarian carcinoma. In some embodiments, the ovarian cancer is high-grade serous ovarian cancer. In some embodiments, the ovarian cancer is high-grade serous ovarian adenocarcinoma. In some embodiments, the ovarian cancer is characterized by mutation of the TP53 gene. In some embodiments, the administration of carfilzomib and vandetanib is simultaneous. In some embodiments, the administration of carfilzomib and vandetanib is sequential.

### BRIEF DESCRIPTION OF THE DRAWINGS

FIG. 1 shows a set of dose-response curves for binimetinib and copanlisib.

FIG. 2 shows a combination dose-response matrix for binimetinib and copanlisib.

FIG. 3 shows a 2D synergy map for binimetinib and copanlisib with the most synergistic area contained within the white box.

FIG. 4 shows a 3D synergy map for binimetinib and copanlisib.

FIG. 5 shows a set of dose-response curves for cobimetinib and buparlisib.

FIG. 6 shows a combination dose-response matrix for cobimetinib and buparlisib.

FIG. 7 shows a 2D synergy map for cobimetinib and buparlisib with the most synergistic area contained within the white box.

FIG. 8 shows a 3D synergy map for cobimetinib and buparlisib.

FIG. 9 shows a set of dose-response curves for carfilzomib and pazopanib.

FIG. 10 shows a combination dose-response matrix for carfilzomib and pazopanib.

FIG. 11 shows a 2D synergy map for carfilzomib and pazopanib with the most synergistic area contained within the white box.

FIG. 12 shows a 3D synergy map for carfilzomib and pazopanib.

FIG. 13 shows a set of dose-response curves for copanlisib and dasatinib.

FIG. 14 shows a combination dose-response matrix for copanlisib and dasatinib.

FIG. 15 shows a 2D synergy map for copanlisib and dasatinib with the most synergistic area contained within the white box.

FIG. 16 shows a 3D synergy map for copanlisib and dasatinib.

FIG. 17 shows a set of dose-response curves for carfilzomib and vandetanib.

FIG. 18 shows a combination dose-response matrix for carfilzomib and vandetanib.

FIG. 19 shows a 2D synergy map for carfilzomib and vandetanib with the most synergistic area contained within the white box.

FIG. 20 shows a 3D synergy map for carfilzomib and vandetanib.

### DETAILED DESCRIPTION

The disclosure provides combinations of chemotherapeutics effective in the treatment of various cancers. In some embodiments, the cancer to be treated is ovarian cancer. In some embodiments, the cancer is high-grade serous ovarian cancer. In some embodiments, the cancer is high-grade serous ovarian adenocarcinoma. In some embodiments, the cancer is ovarian cancer characterized by mutation of the TP53 gene.

### Definitions

It is to be understood that the terminology used herein is for the purpose of describing particular embodiments only, and is not intended to be limiting. Unless defined otherwise, all technical and scientific terms used herein have the same meaning as commonly understood by one of ordinary skill in the art to which the invention pertains. Although other methods, systems, and networks similar, or equivalent, to those described herein can be used in the practice of the present invention, the preferred materials and methods are described herein.

In describing and claiming the present invention, the following terminology will be used in accordance with the definitions set out below.

As used in this specification and the appended claims, the singular forms “a,” “an,” and “the” include plural referents unless the context clearly dictates otherwise. Thus, for example, reference to “an excipient” includes a combination of two or more such excipients, reference to “an active pharmaceutical ingredient” includes one or more active pharmaceutical ingredients, and the like. Unless specifically stated or obvious from context, as used herein, the term “or” is understood to be inclusive and covers both “or” and “and.”

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 5% or less in either direction (greater than or less than) of the stated reference value unless otherwise stated or otherwise evident from the context (except where such number would exceed 100% of a possible value).

The terms “subject” and “patient” as used herein refers to any member of the subphylum Chordata, including, without limitation, humans and other primates, including non-human primates such as rhesus macaques and other monkey species and chimpanzees and other ape species; farm animals such as cattle, sheep, pigs, goats, and horses; domestic mammals such as dogs and cats; laboratory animals including rodents such as mice, rats, and guinea pigs; birds, including domestic, wild, and game birds such as chickens, turkeys, and other gallinaceous birds, ducks, geese, and the like. The term does not denote a particular age or gender. Thus, both adult and newborn individuals are intended to be covered.

The terms “administer” and “administering” as used herein refer to providing a therapeutic to a subject. Multiple techniques of administering a therapeutic exist in the art including, but not limited to, intravenous, oral, aerosol, parenteral, ophthalmic, pulmonary, and topical administration. In some embodiments, administration is oral (e.g., via tablet or capsule). In some embodiments, administration is via injection.

The terms “treatment” and “treating” as used herein refer to amelioration of a disease or disorder, or at least one sign or symptom thereof. “Treatment” or “treating” can refer to reducing the progression of a disease or disorder, as determined by, e.g., stabilization of at least one sign or symptom or a reduction in the rate of progression as determined by a reduction in the rate of progression of at least one sign or symptom.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

The term “therapeutically effective amount” as used herein refers to an amount of a drug, formulation, or composition to achieve a particular biological result. In certain embodiments, a therapeutically effective amount treats or prevents a disease or a disorder, e.g., ameliorates at least one sign or symptom of the disorder. In various embodiments, the disease or disorder is a cancer.

As used herein, the compounds disclosed herein include the pharmaceutical acceptable form thereof. Some examples of the compounds include, but are not limited to, binimetinib, carfilzomib, copanlisib, dasatinib, ibrutinib, ponatinib, and vandetanib. As used herein, a “pharmaceutically acceptable form” of a disclosed compound includes, but is not limited to, pharmaceutically acceptable salts, hydrates, solvates, isomers, prodrugs, esters, and isotopically labeled derivatives of the disclosed compounds. In one embodiment, a “pharmaceutically acceptable form” includes, but is not limited to, pharmaceutically acceptable salts, isomers, and prodrugs.

In some embodiment, the pharmaceutically acceptable form is a pharmaceutically acceptable salt. The term “pharmaceutically acceptable salt(s)” used herein refers to salts of acidic or basic groups that may be present in compounds used in the present compositions. Examples of salts (or salt forms) include, but are not limited to, mineral or organic acid salts of basic residues such as amines, alkali or organic salts of acidic residues such as carboxylic acids, and the like. Lists of suitable salts are found in Remington's Pharmaceutical Sciences, 17th ed., Mack Publishing Company, Easton, Pa., 1985, p. 1418, the disclosure of which is hereby incorporated by reference in its entirety.

In some embodiment, the pharmaceutically acceptable form is a solvate (e.g., a hydrate). As used herein, the term “solvate” refers to compounds that further include a stoichiometric or non-stoichiometric amount of solvent bound by non-covalent intermolecular forces. The solvate may be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or one to about 2, about 3 or about 4, solvent or water molecules.

In some embodiment, the pharmaceutically acceptable form is a prodrug. As used herein, the term “prodrug” refers to compounds that are transformed in vivo to yield a disclosed compound or a pharmaceutically acceptable form of the compound. A prodrug may be inactive when administered to a subject, but is converted in vivo to an active compound, for example, by hydrolysis (e.g., hydrolysis in blood). In certain cases, a prodrug has improved physical and/or delivery properties over the parent compound. Prodrugs are typically designed to enhance pharmaceutically and/or pharmacokinetically based properties associated with the parent compound. Exemplary advantages of a prodrug can include, but are not limited to, its physical properties, such as enhanced water solubility for parenteral administration at physiological pH compared to the parent compound, or it enhances absorption from the digestive tract, or it can enhance drug stability for long-term storage.

The term “prodrug” is also meant to include any covalently bonded carriers, which release the active compound in vivo when such prodrug is administered to a subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

In some embodiment, the pharmaceutically acceptable form is an isomer. “Isomers” are different compounds that have the same molecular formula. “Stereoisomers” are isomers that differ only in the way the atoms are arranged in space. As used herein, the term “isomer” includes any and all geometric isomers and stereoisomers. For example, “isomers” include geometric double bond cis- and trans-isomers, also termed E- and Z-isomers; R- and S-enantiomers; diastereomers, (d)-isomers and (I)-isomers, racemic mixtures thereof; and other mixtures thereof, as falling within the scope of this disclosure.

The term “synergy” or “synergistic” encompasses the interaction or cooperation of two or more substances to produce a combined effect greater than the sum of their separate effects. In certain embodiments, synergy or synergistic effect refers to an advantageous effect of using two or more agents in combination, e.g., in a pharmaceutical composition, or in a method of treatment. Several models exist to measure synergy including the highest single agent model (HSA), the Loewe additivity model, and the Bliss independence model, discussed in Berenbaum MC. Pharmacol Rev. 1989; 41:93-141; Loewe S. Arzneimittel-forschung. 1953; 3:285-290 and Bliss CI. Ann Appl Biol. 1939; 26:585-615, incorporated by reference herein in their entireties. Any method for measuring synergy can be used.

In some embodiments, the synergistic effect is that a lower dosage of one or both of the agents is needed to achieve an effect. For example, the combination can provide a selected effect, e.g., a therapeutic effect, when at least one of the agents is administered at a lower dosage than the dose of that agent that would be required to achieve the same therapeutic effect when the agent is administered as a monotherapy. In certain embodiments, the combination of a first agent and a second agent allows the first agent to be administered at a lower dosage than would be required to achieve the same therapeutic effect if the first agent is administered as a monotherapy.

In some embodiments, the synergistic effect is a reduction, prevention, delay, or decrease in the occurrence or the likelihood of occurrence of one or more side effects, toxicity, resistance, that would otherwise be associated with administration of at least one of the agents.

In some embodiments, the synergistic effect is a reduction in resistance (e.g., a decrease in a measure of resistance or a decreased likelihood of developing resistance), or a delay in the development of resistance, to at least one of the agents.

As used herein, “combinations,” “combination therapy,” or “in combination with” refer to the use of more than one compound or agent to treat a particular disorder or condition. For example, Compound 1 may be administered in combination with at least one additional therapeutic agent. By “in combination with,” it is not intended to imply that the other therapy and Compound 1 must be administered at the same time and/or formulated for delivery together, although these methods of delivery are within the scope of this disclosure. Compound 1 can be administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), one or more other additional agents. In general, each therapeutic agent will be administered at a dose and/or on a time schedule determined for that particular agent. The other therapeutic agent can be administered with Compound 1 herein in a single composition or separately in a different composition.

The term “cancer” includes, but is not limited to, solid tumors and blood born tumors. The term “cancer” refers to disease of skin tissues, organs, blood, and vessels, including, but not limited to, cancers of the bladder, bone or blood, brain, breast, cervix, chest, colon, endometrium, esophagus, eye, head, kidney, liver, lymph nodes, lung, mouth, neck, ovaries, pancreas, prostate, rectum, stomach, testis, throat, and uterus.

Avapritinib is an orally bioavailable, small molecule inhibitor of tyrosine kinase platelet derived growth factor alpha (PDGFRA) with potential antineoplastic activity. Avapritinib is also known as BLU-285. Avapritinib has the chemical name (S)-1-(4-fluorophenyl)-1-(2-(4-(6-(1-methyl-1H-pyrazol-4-yl)pyrrolo[2,1-f][1,2,4]triazin-4-yl)piperazin-1-yl)pyrimidin-5-yl)ethan-1-amine, and the following structure:

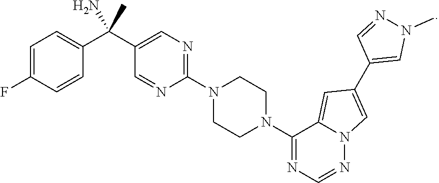 [SMILES: Cn1cc(-c2cc3c(N4CCN(c5ncc([C@@](C)(N)c6ccc(F)cc6)cn5)CC4)ncnn3c2)cn1]

Binimetinib is an orally bioavailable inhibitor of mitogen-activated protein kinase kinase (MEK) with potential antineoplastic activity. Binimetinib is also known as Mektovi and ARRY-162. Binimetinib has the chemical name 5-((4-bromo-2-fluorophenyl)amino)-4-fluoro-N-(2-hydroxyethoxy)-1-methyl-1H-benzo[d]imidazole-6-carboxamide, and the following structure:

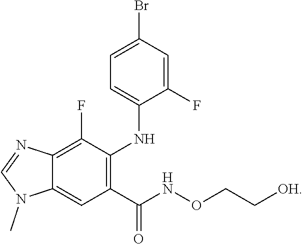 [SMILES: Cn1cnc2c(F)c(Nc3ccc(Br)cc3F)c(C(=O)NOCCO)cc21]

Buparlisib is an investigational, small molecule pan-class I phosphoinositide 3-kinase (PI3K) inhibitor with potential antineoplastic activity. Buparlisib is also known as BKM120. Buparlisib has the chemical name 5-(2,6-dimorpholinopyrimidin-4-yl)-4-(trifluoromethyl)pyridin-2-amine, and the following structure:

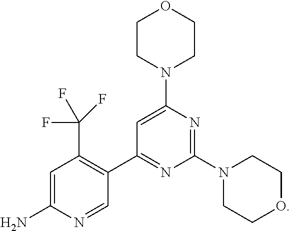 [SMILES: Nc1cc(C(F)(F)F)c(-c2cc(N3CCOCC3)nc(N3CCOCC3)n2)cn1]

Cabozantinib is an orally bioavailable, small molecule inhibitor of the tyrosine kinases c-Met and VEGFR2 with potential antineoplastic activity. Cabozantinib is also known as XL184 and BMS907351. Cabozantinib has the chemical name N-(4-((6,7-dimethoxyquinolin-4-yl)oxy)phenyl)-N-(4-fluorophenyl)cyclopropane-1,1-dicarboxamide, and the following structure:

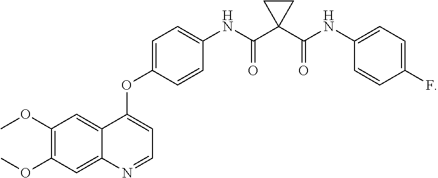 [SMILES: COc1cc2nccc(Oc3ccc(NC(=O)C4(C(=O)Nc5ccc(F)cc5)CC4)cc3)c2cc1OC]

Carfilzomib is an injectable, small molecule inhibitor of the 20S proteosome and of the ubiquitin-proteasome pathway with potential antineoplastic activity. Carfilzomib is also known as PX-171. Carfilzomib has the chemical name (S)-4-methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)pentanamide, and the following structure:

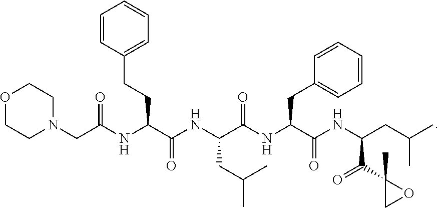 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

Cobimetinib is an orally bioavailable, small molecule inhibitor of mitogen-activated protein kinase kinase (MEK) with potential antineoplastic activity. Cobimetinib is also known as GDC-0973 and XL-518. Cobimetinib has the chemical name (S)-(3,4-difluoro-2-((2-fluoro-4-iodophenyl)amino)phenyl)(3-hydroxy-3-(piperidin-2-yl)azetidin-1-yl)methanone, and the following structure:

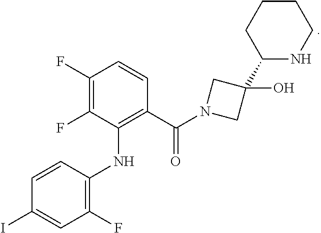 [SMILES: O=C(c1ccc(F)c(F)c1Nc1ccc(I)cc1F)N1CC(O)([C@@H]2CCCCN2)C1]

Copanlisib is an injectable, small molecule inhibitor of phosphatidylinositol-3-kinase (PI3K) with potential antineoplastic activity. Copanlisib is also known as BAY 80-6946. Copanlisib has the chemical name 2-amino-N-(7-methoxy-8-(3-morpholinopropoxy)-2,3-dihydroimidazo[1,2-c]quinazolin-5-yl)pyrimidine-5-carboxamide, and the following structure:

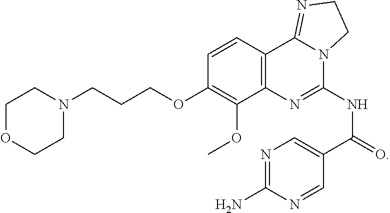 [SMILES: COc1c(OCCCN2CCOCC2)ccc2c1N=C(NC(=O)c1cnc(N)nc1)N1CCN=C21]

Dasatinib is an orally bioavailable, small molecule multiple kinase inhibitor, with targets including BCR/Abl, Src, and c-Kit. The compound has potential antineoplastic activity and is also known as BMS-354825. Dasatinib has the chemical name N-(2-chloro-6-methylphenyl)-2-((6-(4-(2-hydroxyethyl)piperazin-1-yl)-2-methylpyrimidin-4-yl)amino)thiazole-5-carboxamide, and the following structure:

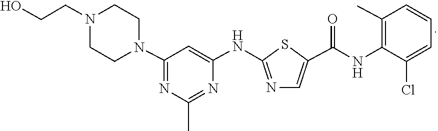 [SMILES: Cc1nc(Nc2ncc(C(=O)Nc3c(C)cccc3Cl)s2)cc(N2CCN(CCO)CC2)n1]

Erlotinib is an orally bioavailable, small molecule inhibitor of epidermal growth factor receptor (EGFR) with potential antineoplastic activity. Erlotinib has the chemical name N-(3-ethynylphenyl)-6,7-bis(2-methoxyethoxy)quinazolin-4-amine, and the following structure:

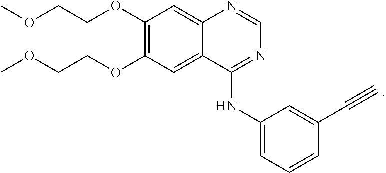 [SMILES: C#Cc1cccc(Nc2ncnc3cc(OCCOC)c(OCCOC)cc23)c1]

Gilteritinib is an orally bioavailable, small molecule inhibitor of FLT3 and AXL with potential antineoplastic activity. Gilteritinib is also known as ASP2215. Gilteritinib has the chemical name 6-ethyl-3-((3-methoxy-4-(4-(4-methylpiperazin-1-yl)piperidin-1-yl)phenyl)amino)-5-((tetrahydro-2H-pyran-4-yl)amino)pyrazine-2-carboxamide, the following structure:

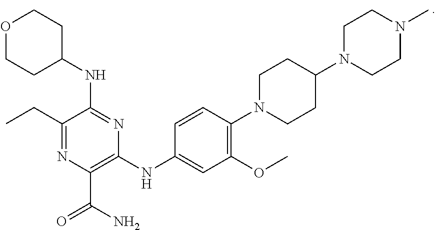 [SMILES: CCc1nc(C(N)=O)c(Nc2ccc(N3CCC(N4CCN(C)CC4)CC3)c(OC)c2)nc1NC1CCOCC1]

Ibrutinib is an orally bioavailable, small molecule inhibitor of Bruton's tyrosine Kinase (BTK) with potential antineoplastic activity. Ibrutinib is also known as PCI-32765 and CRA-032765. Ibrutinib has the chemical name (R)-1-(3-(4-amino-3-(4-phenoxyphenyl)-1H-pyrazolo[3,4-d]pyrimidin-1-yl)piperidin-1-yl)prop-2-en-1-one, and the following structure:

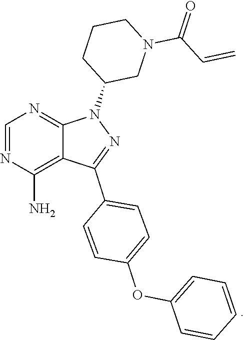 [SMILES: C=CC(=O)N1CCC[C@@H](n2nc(-c3ccc(Oc4ccccc4)cc3)c3c(N)ncnc32)C1]

Larotrectinib is an orally bioavailable, small molecule inhibitor of tropmyosin receptor kinase (Trk) with potential antineoplastic activity. Larotrectinib is also known as LOXO-101 and ARRY-470. Larotrectinib has the chemical name (S)—N-(5-((R)-2-(2,5-difluorophenyl)pyrrolidin-1-yl)pyrazolo[1,5-a]pyrimidin-3-yl)-3-hydroxypyrrolidine-1-carboxamide, and the following structure:

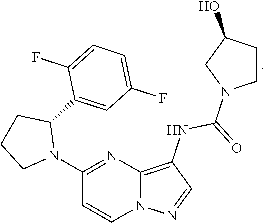 [SMILES: O=C(Nc1cnn2ccc(N3CCC[C@@H]3c3cc(F)ccc3F)nc12)N1CC[C@H](O)C1]

Palbociclib is an orally bioavailable, small molecule inhibitor of the cyclin-dependent kinases CDK4 and CDK6 with potential antineoplastic activity. Palbociclib is also known as PD-0332991. Palbociclib has the chemical name 6-acetyl-8-cyclopentyl-5-methyl-2-((5-(piperazin-1-yl)pyridin-2-yl)amino)pyrido[2,3-d]pyrimidin-7(8H)-one, and the following structure:

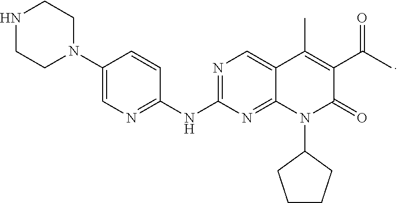 [SMILES: CC(=O)c1c(C)c2cnc(Nc3ccc(N4CCNCC4)cn3)nc2n(C2CCCC2)c1=O]

Pazopanib is an orally bioavailable, small molecule multiple kinase inhibitor, with targets including vascular endothelial growth factor receptor (VEGFR), platelet-derived growth factor receptor (PDGFR), c-KIT, and FGFR. The compound is a potential antineoplastic agent. Pazopanib has the chemical name 5-((4-((2,3-dimethyl-2H-indazol-6-yl)(methyl)amino)pyrimidin-2-yl)amino)-2-methylbenzenesulfonamide, and the following structure:

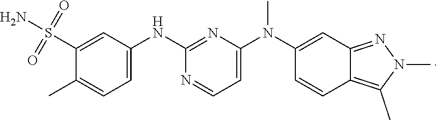 [SMILES: Cc1ccc(Nc2nccc(N(C)c3ccc4c(C)n(C)nc4c3)n2)cc1S(N)(=O)=O]

Pexidartinib is an orally bioavailable, small molecule colony-stimulating factor (CSF-1) receptor pathway inhibitor with potential antineoplastic activity. Pexidartinib is also known as PLX-3397. Pexidartinib has the chemical name 5-((5-chloro-1H-pyrrolo[2,3-b]pyridin-3-yl)methyl)-N-((6-(trifluoromethyl)pyridin-3-yl)methyl)pyridin-2-amine, and the following structure:

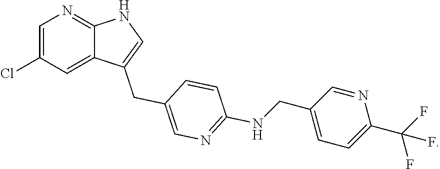 [SMILES: FC(F)(F)c1ccc(CNc2ccc(Cc3c[nH]c4ncc(Cl)cc34)cn2)cn1]

Ponatinib is an orally bioavailable, small molecule multiple kinase inhibitor, with targets including BCR-ABL and the PI3K/AKT/mTOR pathway. The compound has potential antineoplastic activity and is also known as AP24534. Ponatinib has the chemical name 3-(imidazo[1,2-b]pyridazin-3-ylethynyl)-4-methyl-N-(4-((4-methylpiperazin-1-yl)methyl)-3-(trifluoromethyl)phenyl)benzamide, and following structure:

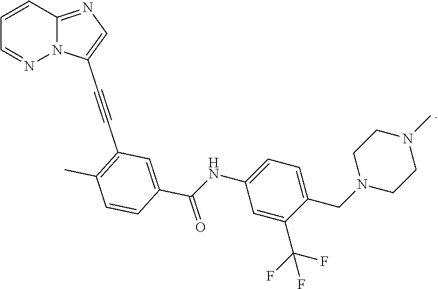 [SMILES: Cc1ccc(C(=O)Nc2ccc(CN3CCN(C)CC3)c(C(F)(F)F)c2)cc1C#Cc1cnc2cccnn12]

Regorafenib is an orally bioavailable, small molecule multiple kinase inhibitor, with targets including VEGFR. The compound has potential antineoplastic activity and is also known as BAY 73-4506. Regorafenib has the chemical name 4-(4-(3-(4-chloro-3-(trifluoromethyl)phenyl)ureido)-3-fluorophenoxy)-N-methylpicolinamide, and the following structure:

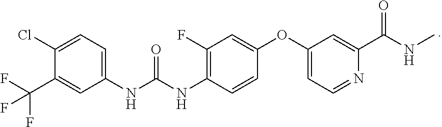 [SMILES: CNC(=O)c1cc(Oc2ccc(NC(=O)Nc3ccc(Cl)c(C(F)(F)F)c3)c(F)c2)ccn1]

Sunitinib is an orally bioavailable, small molecule multiple kinase inhibitor, with targets including platelet-derived growth factor receptors (PDGF-R), vascular endothelial growth factor receptors (VEGF-R), and c-KIT (CD117). The compound has potential antineoplastic activity and is also known as SU11248. Sunitinib has the chemical name (Z)—N-(2-(diethylamino)ethyl)-5-((5-fluoro-2-oxoindolin-3-ylidene)methyl)-2,4-dimethyl-1H-pyrrole-3-carboxamide, and the following structure:

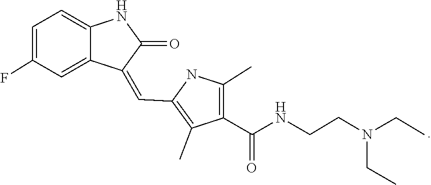

Vandetanib is an orally bioavailable, small molecule inhibitor of vascular endothelial growth factor receptor (VEGFR), epidermal growth factor receptor (EGFR), and RET-tyrosine kinase with potential antineoplastic activity. Vandetanib is also known as ZD6474. Vandetanib has the chemical name N-(4-bromo-2-fluorophenyl)-6-methoxy-7-((1-methylpiperidin-4-yl)methoxy)quinazolin-4-amine, and the following structure:

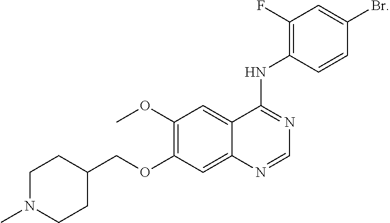 [SMILES: COc1cc2c(Nc3ccc(Br)cc3F)ncnc2cc1OCC1CCN(C)CC1]

### General Overview of the Invention

Complex diseases, such as cancers, are caused by multiple interacting pathogenic genes. As a result, the traditional “one target, one drug” therapeutic mode has limited potential. Recent studies have shown that targeting multiple disease-associated genes has greater therapeutic potential than single-target therapies, and may not only bring synergistic or additive effects, but also reduced toxicity and delayed resistance for effective disease control. Inhibitors targeting synthetic lethal partners of genes mutated in tumors are already successfully utilized for effective and specific treatment in the clinic.

The present embodiments include therapeutic combinations and methods for treating ovarian cancer that were identified using pattern-discovery techniques described in U.S. patent application Ser. No. 17/117,742 (titled “Systems and Methods for Pattern Discovery in Sparse Unlabeled Data” and filed Dec. 20, 2020), which is incorporated herein by reference in its entirety. The identification of these therapeutic combinations started with a genomics data set organized as a matrix in which each row corresponded to a patient, each column corresponded to a gene, and each matrix element stored a gene-expression level. Outlier gene-expression levels were identified for each gene, from which a set of outlier genes was constructed. Pairs of outlier genes were then used to train a skip-gram model to embed the outlier genes in a high-dimensionality feature space. The output of the skip-gram model was a weight matrix of the hidden layer of the skip-gram model. The weight matrix was separated into columns, where each column was a feature vector that located a corresponding outlier gene in the high-dimensionality feature space. Cosine similarities were then calculated between all pairs of outlier genes to quantify how strongly both genes in the pair were upregulated (i.e., overexpressed). The gene pairs were ranked based on their cosine similarities, and a gene-pair list was then constructed by keeping only the highest-ranked gene pairs (i.e., gene pairs for which both of the genes' expressions were significantly upregulated) for which both of the genes are druggable. Drugs targeting each gene on the gene-pair list were then identified using information culled from public databases, as described in more detail in the above referenced disclosure.

Based on the ovarian cancer data set, the following outlier gene-pairs, and corresponding drugs, were identified as feasible candidates for pharmaceutical combinations to treat ovarian cancer. In the following table, each row represents two drugs that, in combination, represent one such pharmaceutical combination. Five of these drug combinations, indicated with asterisks, were observed to exhibit synergistic inhibition of ovarian tumor cell growth.

| Gene1 | Drug1 | Gene2 | Drug2 |
|---|---|---|---|
| MAP2K1* | Binimetinib* | PIK3CB* | Copanlisib* |
| MAP2K1* | Cobimetinib* | PIK3CB* | Buparlisib* |
| MAP2K1 | Binimetinib | PDGFRA | Ponatinib |
| PDGFRA | Ponatinib | PIK3CB | Copanlisib |
| PDGFRA | Avapritinib | PIK3CB | Buparlisib |
| CDK6 | Palbociclib | FLT1 | Sunitinib |
| AXL | Gilteritinib | NTRK2 | Larotrectinib |
| PIK3R5 | Copanlisib | CSF1R | Pazopanib |
| PIK3R5 | Copanlisib | CSF1R | Pexidartinib |
| CSF1R | Pazopanib | AXL | Gilteritinib |
| MAP2K1 | Binimetinib | PTK6 | Ibrutinib |
| PDGFRA | Ponatinib | PTK6 | Ibrutinib |
| PIK3CB | Copanlisib | PTK6 | Ibrutinib |
| PDGFRA | Ponatinib | SRMS | Dasatinib |
| PSMC2 | Carfilzomib | AXL | Gilteritinib |
| EGFR | Erlotinib | PSMD11 | Carfilzomib |
| PSMC2 | Carfilzomib | PDGFRA | Ponatinib |
| HCK | Dasatinib | PSMC2 | Carfilzomib |
| PSMC2* | Carfilzomib* | CSF1R* | Pazopanib* |
| MAP2K1 | Binimetinib | PSMC4 | Carfilzomib |
| PSMC4 | Carfilzomib | PTK6 | Ibrutinib |
| KDR | Cabozantinib | PSMC4 | Carfilzomib |
| CDK6 | Palbociclib | EPHB4 | Vandetanib |
| EPHA1 | Vandetanib | PDGFRA | Ponatinib |
| EPHA1 | Vandetanib | PIK3CB | Copanlisib |
| MAPK11 | Regorafenib | SRC | Dasatinib |
| MAPK11 | Regorafenib | PIK3CB | Copanlisib |
| MAP2K1 | Binimetinib | SRMS | Dasatinib |
| PIK3CB* | Copanlisib* | SRMS* | Dasatinib* |
| PSMC4* | Carfilzomib* | EPHA5* | Vandetanib* |
| EPHA4 | Vandetanib | NTRK2 | Larotrectinib |

Accordingly, in one aspect, the present disclosure provides a pharmaceutical combination including a therapeutically effective amount of binimetinib and a therapeutically effective amount of copanlisib. In one embodiment, binimetinib and copanlisib are provided in synergistically effective amounts.

In some embodiments, the molar ratio of binimetinib to copanlisib is between about 15:1 to about 55:1 or the molar ratio is any particular value within this range. In some embodiments, the molar ratio of binimetinib to copanlisib is between about 23:1 to about 43:1. In some embodiments, the molar ratio of binimetinib to copanlisib is between about 28:1 to about 38:1. In some embodiments, the molar ratio of binimetinib to copanlisib is about 28:1, about 29:1, about 30:1, about 31:1, about 32:1, about 33:1, about 34:1, about 35:1, about 36:1, about 37:1, or about 38:1. In some embodiments, the molar ratio of binimetinib to copanlisib is about 33:1.

In some embodiments, the molar ratio of binimetinib to copanlisib is between about 1:1 and about 1:10 or the molar ratio is any particular value within this range. In some embodiments, the molar ratio of binimetinib to copanlisib is between about 1:4 to about 1:10. In some embodiments, the molar ratio of binimetinib to copanlisib is about 1:4, about 1:5, about 1:6, about 1:7, about 1:8, about 1:9, or about 1:10. In some embodiments, the molar ratio of binimetinib to copanlisib is between about 1:6 to about 1:8. In some embodiments, the molar ratio of binimetinib to copanlisib is about 1.7.

In some embodiments, the binimetinib is administered in an amount between about 0.1 mg/kg to about 30 mg/kg or at any particular dose within this range. In some embodiments, the binimetinib is administered in an amount between about 0.1 mg/kg to about 20 mg/kg. In some embodiments, the binimetinib is administered in an amount between about 0.1 mg/kg to about 15 mg/kg. In some embodiments, the binimetinib is administered in an amount between about 0.1 mg/kg to about 5 mg/kg. In some embodiments, the binimetinib is administered in an amount between about 5 mg/kg to about 10 mg/kg. In some embodiments, the binimetinib is administered in an amount between about 10 mg/kg to about 15 mg/kg. In some embodiments, the binimetinib is administered in an amount between about 15 mg/kg to about 20 mg/kg. In some embodiments, the binimetinib is administered in an amount between about 20 mg/kg to about 25 mg/kg. In some embodiments, the binimetinib is administered in an amount between about 25 mg/kg to about 30 mg/kg.

In some embodiments, the copanlisib is administered in an amount between about 0.1 mg/kg to about 14 mg/kg or at any particular dose within this range. In some embodiments, the copanlisib is administered in an amount between about 0.1 mg/kg to about 7 mg/kg. In some embodiments, the copanlisib is administered in an amount between about 0.1 mg/kg to about 5 mg/kg. In some embodiments, the copanlisib is administered in an amount between about 5 mg/kg to about 10 mg/kg. In some embodiments, the copanlisib is administered in an amount between about 10 mg/kg to about 14 mg/kg. In some embodiments, the copanlisib is administered in an amount between about 0.1 mg/kg to about 1 mg/kg. In some embodiments, the copanlisib is administered in an amount between about 1 mg/kg to about 2 mg/kg. In some embodiments, the copanlisib is administered in an amount between about 2 mg/kg to about 3 mg/kg. In some embodiments, the copanlisib is administered in an amount between about 3 mg/kg to about 4 mg/kg. In some embodiments, the copanlisib is administered in an amount between about 4 mg/kg to about 5 mg/kg. In some embodiments, the copanlisib is administered in an amount between about 5 mg/kg to about 6 mg/kg. In some embodiments, the copanlisib is administered in an amount between about 6 mg/kg to about 7 mg/kg. In some embodiments, the copanlisib is administered in an amount between about 7 mg/kg to about 8 mg/kg. In some embodiments, the copanlisib is administered in an amount between about 8 mg/kg to about 9 mg/kg. In some embodiments, the copanlisib is administered in an amount between about 9 mg/kg to about 10 mg/kg. In some embodiments, the copanlisib is administered in an amount between about 10 mg/kg to about 11 mg/kg. In some embodiments, the copanlisib is administered in an amount between about 11 mg/kg to about 12 mg/kg. In some embodiments, the copanlisib is administered in an amount between about 12 mg/kg to about 13 mg/kg. In some embodiments, the copanlisib is administered in an amount between about 13 mg/kg to about 14 mg/kg.

In some embodiments, the combination of binimetinib and copanlisib is formulated as a single pharmaceutical composition. Accordingly, in some embodiments, the present disclosure provides a pharmaceutical composition comprising binimetinib, copanlisib, and a pharmaceutically acceptable carrier. In some embodiments, the combination of binimetinib and copanlisib is formulated as separate pharmaceutical compositions. Accordingly, in some embodiments, the present disclosure provides (1) a pharmaceutical composition comprising binimetinib and a pharmaceutically acceptable carrier and (2) a pharmaceutical composition comprising copanlisib and a pharmaceutically acceptable carrier.

The present disclosure also provides a kit comprising the combination of binimetinib and copanlisib. In some embodiments, the kit comprises instructions for the administration of the combination.

In another aspect, the present disclosure provides a method of treating cancer in a subject in need thereof, including administering to the subject a therapeutically effective amount of binimetinib and a therapeutically effective amount of copanlisib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene. In some embodiments, the administration of binimetinib and copanlisib is simultaneous. In some embodiments, the administration of binimetinib and copanlisib is sequential.

In one aspect, the present disclosure provides a pharmaceutical combination including a therapeutically effective amount of cobimetinib and a therapeutically effective amount of buparlisib. In one embodiment, cobimetinib and buparlisib are provided in synergistically effective amounts.

In some embodiments, the molar ratio of cobimetinib to buparlisib is between about 15:1 to about 55:1 or the molar ratio is any particular value within this range. In some embodiments, the molar ratio of cobimetinib to buparlisib is between about 23:1 to about 43:1. In some embodiments, the molar ratio of cobimetinib to buparlisib is between about 28:1 to about 38:1. In some embodiments, the molar ratio of cobimetinib to buparlisib is about 28:1, about 29:1, about 30:1, about 31:1, about 32:1, about 33:1, about 34:1, about 35:1, about 36:1, about 37:1, or about 38:1. In some embodiments, the molar ratio of cobimetinib to buparlisib is about 33:1.

In some embodiments, the molar ratio of cobimetinib to buparlisib is between about 1:1 to about 20:1 or the molar ratio is any particular value within this range. In some embodiments, the molar ratio of cobimetinib to buparlisib is between about 5:1 to about 15:1. In some embodiments, the molar ratio of cobimetinib to buparlisib is between about 8:1 to about 12:1. In some embodiments, the molar ratio of cobimetinib to buparlisib is about 5:1, about 6:1, about 7:1, about 8:1, about 9:1, about 10:1, about 11:1, about 12:1, about 13:1, about 14:1, about 15:1, In some embodiments, the molar ratio of cobimetinib to buparlisib is about 10:1.

In some embodiments, the cobimetinib is administered in an amount between about 0.1 mg/kg to about 5 mg/kg or at any particular dose within this range. In some embodiments, the cobimetinib is administered in an amount between about 0.1 mg/kg to about 2.5 mg/kg. In some embodiments, the cobimetinib is administered in an amount between about 0.1 mg/kg to about 0.5 mg/kg. In some embodiments, the cobimetinib is administered in an amount between about 0.5 mg/kg to about 1 mg/kg. In some embodiments, the cobimetinib is administered in an amount between about 1 mg/kg to about 1.5 mg/kg. In some embodiments, the cobimetinib is administered in an amount between about 1.5 mg/kg to about 2 mg/kg. In some embodiments, the cobimetinib is administered in an amount between about 2 mg/kg to about 2.5 mg/kg. In some embodiments, the cobimetinib is administered in an amount between about 2.5 mg/kg to about 3 mg/kg. In some embodiments, the cobimetinib is administered in an amount between about 3 mg/kg to about 3.5 mg/kg. In some embodiments, the cobimetinib is administered in an amount between about 3.5 mg/kg to about 4 mg/kg. In some embodiments, the cobimetinib is administered in an amount between about 4 mg/kg to about 4.5 mg/kg. In some embodiments, the cobimetinib is administered in an amount between about 4.5 mg/kg to about 5 mg/kg.

In some embodiments, the buparlisib is administered in an amount between about 0.1 mg/kg to about 50 mg/kg or at any particular dose within this range. In some embodiments, the buparlisib is administered in an amount between about 0.1 mg/kg to about 25 mg/kg. In some embodiments, the buparlisib is administered in an amount between about 0.1 mg/kg to about 20 mg/kg. In some embodiments, the buparlisib is administered in an amount between about 15 mg/kg to about 35 mg/kg. In some embodiments, the buparlisib is administered in an amount between about 35 mg/kg to about 50 mg/kg. In some embodiments, the buparlisib is administered in an amount between about 0.1 mg/kg to about 5 mg/kg. In some embodiments, the buparlisib is administered in an amount between about 5 mg/kg to about 10 mg/kg. In some embodiments, the buparlisib is administered in an amount between about 10 mg/kg to about 15 mg/kg. In some embodiments, the buparlisib is administered in an amount between about 15 mg/kg to about 20 mg/kg. In some embodiments, the buparlisib is administered in an amount between about 20 mg/kg to about 25 mg/kg. In some embodiments, the buparlisib is administered in an amount between about 25 mg/kg to about 30 mg/kg. In some embodiments, the buparlisib is administered in an amount between about 30 mg/kg to about 35 mg/kg. In some embodiments, the buparlisib is administered in an amount between about 35 mg/kg to about 40 mg/kg. In some embodiments, the buparlisib is administered in an amount between about 40 mg/kg to about 45 mg/kg. In some embodiments, the buparlisib is administered in an amount between about 45 mg/kg to about 50 mg/kg.

In some embodiments, the combination of cobimetinib and buparlisib is formulated as a single pharmaceutical composition. Accordingly, in some embodiments, the present disclosure provides a pharmaceutical composition comprising cobimetinib, buparlisib, and a pharmaceutically acceptable carrier. In some embodiments, the combination of cobimetinib and buparlisib is formulated as separate pharmaceutical compositions. Accordingly, in some embodiments, the present disclosure provides (1) a pharmaceutical composition comprising cobimetinib and a pharmaceutically acceptable carrier and (2) a pharmaceutical composition comprising buparlisib and a pharmaceutically acceptable carrier.

The present disclosure also provides a kit comprising the combination of cobimetinib and buparlisib. In some embodiments, the kit comprises instructions for the administration of the combination.

In another aspect, the present disclosure provides a method of treating cancer in a subject in need thereof, including administering to the subject a therapeutically effective amount of cobimetinib and a therapeutically effective amount of buparlisib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene. In some embodiments, the administration of cobimetinib and buparlisib is simultaneous. In some embodiments, the administration of cobimetinib and buparlisib is sequential.

In one aspect, the present disclosure provides a pharmaceutical combination including a therapeutically effective amount of carfilzomib and a therapeutically effective amount of pazopanib. In one embodiment, carfilzomib and pazopanib are provided in synergistically effective amounts.

In some embodiments, the molar ratio of carfilzomib to pazopanib is between about 1:1 to about 10:1 or the molar ratio is any particular value within this range. In some embodiments, the molar ratio of carfilzomib to pazopanib is between about 1:1 to about 6:1. In some embodiments, the molar ratio of carfilzomib to pazopanib is between about 2:1 to about 5:1. In some embodiments, the molar ratio of carfilzomib to pazopanib is between about 3:1 to about 4:1. In some embodiments, the molar ratio of carfilzomib to pazopanib is about 1:1, about 1.5:1, about 2:1, about 2.5:1, about 3:1, about 3.5:1, about 4:1, about 4.5:1, about 5:1, about 5.5:1, or about 6:1. In some embodiments, the molar ratio of carfilzomib to pazopanib is about 3:1. In some embodiments, the molar ratio of carfilzomib to pazopanib is about 4:1.

In some embodiments, the carfilzomib is administered in an amount between about 0.1 mg/kg to about 5 mg/kg or at any particular dose within this range. In some embodiments, the carfilzomib is administered in an amount between about 0.1 mg/kg to about 2.5 mg/kg. In some embodiments, the carfilzomib is administered in an amount between about 0.1 mg/kg to about 0.5 mg/kg. In some embodiments, the carfilzomib is administered in an amount between about 0.5 mg/kg to about 1 mg/kg. In some embodiments, the carfilzomib is administered in an amount between about 1 mg/kg to about 1.5 mg/kg. In some embodiments, the carfilzomib is administered in an amount between about 1.5 mg/kg to about 2 mg/kg. In some embodiments, the carfilzomib is administered in an amount between about 2 mg/kg to about 2.5 mg/kg. In some embodiments, the carfilzomib is administered in an amount between about 2.5 mg/kg to about 3 mg/kg. In some embodiments, the carfilzomib is administered in an amount between about 3 mg/kg to about 3.5 mg/kg. In some embodiments, the carfilzomib is administered in an amount between about 3.5 mg/kg to about 4 mg/kg. In some embodiments, the carfilzomib is administered in an amount between about 4 mg/kg to about 4.5 mg/kg. In some embodiments, the carfilzomib is administered in an amount between about 4.5 mg/kg to about 5 mg/kg.

In some embodiments, the pazopanib is administered in an amount between about 0.1 mg/kg to about 30 mg/kg or at any particular dose within this range. In some embodiments, the pazopanib is administered in an amount between about 0.1 mg/kg to about 20 mg/kg. In some embodiments, the pazopanib is administered in an amount between about 0.1 mg/kg to about 15 mg/kg. In some embodiments, the pazopanib is administered in an amount between about 0.1 mg/kg to about 5 mg/kg. In some embodiments, the pazopanib is administered in an amount between about 5 mg/kg to about 10 mg/kg. In some embodiments, the pazopanib is administered in an amount between about 10 mg/kg to about 15 mg/kg. In some embodiments, the pazopanib is administered in an amount between about 15 mg/kg to about 20 mg/kg. In some embodiments, the pazopanib is administered in an amount between about 20 mg/kg to about 25 mg/kg. In some embodiments, the pazopanib is administered in an amount between about 25 mg/kg to about 30 mg/kg.

In some embodiments, the combination of carfilzomib and pazopanib is formulated as a single pharmaceutical composition. Accordingly, in some embodiments, the present disclosure provides a pharmaceutical composition comprising carfilzomib, pazopanib, and a pharmaceutically acceptable carrier. In some embodiments, the combination of carfilzomib and pazopanib is formulated as separate pharmaceutical compositions. Accordingly, in some embodiments, the present disclosure provides (1) a pharmaceutical composition comprising carfilzomib and a pharmaceutically acceptable carrier and (2) a pharmaceutical composition comprising pazopanib and a pharmaceutically acceptable carrier.

The present disclosure also provides a kit comprising the combination of carfilzomib and pazopanib. In some embodiments, the kit comprises instructions for the administration of the combination.

In another aspect, the present disclosure provides a method of treating cancer in a subject in need thereof, including administering to the subject a therapeutically effective amount of carfilzomib and a therapeutically effective amount of pazopanib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene. In some embodiments, the administration of carfilzomib and pazopanib is simultaneous. In some embodiments, the administration of carfilzomib and pazopanib is sequential.

In one aspect, the present disclosure provides a pharmaceutical combination including a therapeutically effective amount of copanlisib and a therapeutically effective amount of dasatinib. In one embodiment, copanlisib and dasatinib are provided in synergistically effective amounts.

In some embodiments, the molar ratio of copanlisib to dasatinib is between about 15:1 to about 55:1 or the molar ratio is any particular value within this range. In some embodiments, the molar ratio of copanlisib to dasatinib is between about 23:1 to about 43:1. In some embodiments, the molar ratio of copanlisib to dasatinib is between about 28:1 to about 38:1. In some embodiments, the molar ratio of copanlisib to dasatinib is about 28:1, about 29:1, about 30:1, about 31:1, about 32:1, about 33:1, about 34:1, about 35:1, about 36:1, about 37:1, or about 38:1. In some embodiments, the molar ratio of copanlisib to dasatinib is about 33:1.

In some embodiments, the copanlisib is administered in an amount between about 0.1 mg/kg to about 14 mg/kg or at any particular dose within this range. In some embodiments, the copanlisib is administered in an amount between about 0.1 mg/kg to about 7 mg/kg. In some embodiments, the copanlisib is administered in an amount between about 0.1 mg/kg to about 5 mg/kg. In some embodiments, the copanlisib is administered in an amount between about 5 mg/kg to about 10 mg/kg. In some embodiments, the copanlisib is administered in an amount between about 10 mg/kg to about 14 mg/kg. In some embodiments, the copanlisib is administered in an amount between about 0.1 mg/kg to about 1 mg/kg. In some embodiments, the copanlisib is administered in an amount between about 1 mg/kg to about 2 mg/kg. In some embodiments, the copanlisib is administered in an amount between about 2 mg/kg to about 3 mg/kg. In some embodiments, the copanlisib is administered in an amount between about 3 mg/kg to about 4 mg/kg. In some embodiments, the copanlisib is administered in an amount between about 4 mg/kg to about 5 mg/kg. In some embodiments, the copanlisib is administered in an amount between about 5 mg/kg to about 6 mg/kg. In some embodiments, the copanlisib is administered in an amount between about 6 mg/kg to about 7 mg/kg. In some embodiments, the copanlisib is administered in an amount between about 7 mg/kg to about 8 mg/kg. In some embodiments, the copanlisib is administered in an amount between about 8 mg/kg to about 9 mg/kg. In some embodiments, the copanlisib is administered in an amount between about 9 mg/kg to about 10 mg/kg. In some embodiments, the copanlisib is administered in an amount between about 10 mg/kg to about 11 mg/kg. In some embodiments, the copanlisib is administered in an amount between about 11 mg/kg to about 12 mg/kg. In some embodiments, the copanlisib is administered in an amount between about 12 mg/kg to about 13 mg/kg. In some embodiments, the copanlisib is administered in an amount between about 13 mg/kg to about 14 mg/kg.

In some embodiments, the dasatinib is administered in an amount between about 0.1 mg/kg to about 50 mg/kg or at any particular dose within this range. In some embodiments, the dasatinib is administered in an amount between about 0.1 mg/kg to about 25 mg/kg. In some embodiments, the dasatinib is administered in an amount between about 0.1 mg/kg to about 20 mg/kg. In some embodiments, the dasatinib is administered in an amount between about 15 mg/kg to about 35 mg/kg. In some embodiments, the dasatinib is administered in an amount between about 35 mg/kg to about 50 mg/kg. In some embodiments, the dasatinib is administered in an amount between about 0.1 mg/kg to about 5 mg/kg. In some embodiments, the dasatinib is administered in an amount between about 5 mg/kg to about 10 mg/kg. In some embodiments, the dasatinib is administered in an amount between about 10 mg/kg to about 15 mg/kg. In some embodiments, the dasatinib is administered in an amount between about 15 mg/kg to about 20 mg/kg. In some embodiments, the dasatinib is administered in an amount between about 20 mg/kg to about 25 mg/kg. In some embodiments, the dasatinib is administered in an amount between about 25 mg/kg to about 30 mg/kg. In some embodiments, the dasatinib is administered in an amount between about 30 mg/kg to about 35 mg/kg. In some embodiments, the dasatinib is administered in an amount between about 35 mg/kg to about 40 mg/kg. In some embodiments, the dasatinib is administered in an amount between about 40 mg/kg to about 45 mg/kg. In some embodiments, the dasatinib is administered in an amount between about 45 mg/kg to about 50 mg/kg. In some embodiments, the dasatinib is administered in an amount between about 0.1 mg/kg to about 2.5 mg/kg. In some embodiments, the dasatinib is administered in an amount between about 0.1 mg/kg to about 0.5 mg/kg. In some embodiments, the dasatinib is administered in an amount between about 0.5 mg/kg to about 1 mg/kg. In some embodiments, the dasatinib is administered in an amount between about 1 mg/kg to about 1.5 mg/kg. In some embodiments, the dasatinib is administered in an amount between about 1.5 mg/kg to about 2 mg/kg. In some embodiments, the dasatinib is administered in an amount between about 2 mg/kg to about 2.5 mg/kg. In some embodiments, the dasatinib is administered in an amount between about 2.5 mg/kg to about 3 mg/kg. In some embodiments, the dasatinib is administered in an amount between about 3 mg/kg to about 3.5 mg/kg. In some embodiments, the dasatinib is administered in an amount between about 3.5 mg/kg to about 4 mg/kg. In some embodiments, the dasatinib is administered in an amount between about 4 mg/kg to about 4.5 mg/kg. In some embodiments, the dasatinib is administered in an amount between about 4.5 mg/kg to about 5 mg/kg.

In some embodiments, the combination of copanlisib and dasatinib is formulated as a single pharmaceutical composition. Accordingly, in some embodiments, the present disclosure provides a pharmaceutical composition comprising copanlisib, dasatinib, and a pharmaceutically acceptable carrier. In some embodiments, the combination of copanlisib and dasatinib is formulated as separate pharmaceutical compositions. Accordingly, in some embodiments, the present disclosure provides (1) a pharmaceutical composition comprising copanlisib and a pharmaceutically acceptable carrier and (2) a pharmaceutical composition comprising dasatinib and a pharmaceutically acceptable carrier.

The present disclosure also provides a kit comprising the combination of copanlisib and dasatinib. In some embodiments, the kit comprises instructions for the administration of the combination.

In another aspect, the present disclosure provides a method of treating cancer in a subject in need thereof, including administering to the subject a therapeutically effective amount of copanlisib and a therapeutically effective amount of dasatinib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene. In some embodiments, the administration of copanlisib and dasatinib is simultaneous. In some embodiments, the administration of copanlisib and dasatinib is sequential.

In one aspect, the present disclosure provides a pharmaceutical combination including a therapeutically effective amount of carfilzomib and a therapeutically effective amount of vandetanib. In one embodiment, carfilzomib and vandetanib are provided in synergistically effective amounts.

In some embodiments, the molar ratio of carfilzomib to vandetanib is between about 15:1 to about 55:1 or the molar ratio is any particular value within this range. In some embodiments, the molar ratio of carfilzomib to vandetanib is between about 23:1 to about 43:1. In some embodiments, the molar ratio of carfilzomib to vandetanib is between about 28:1 to about 38:1. In some embodiments, the molar ratio of carfilzomib to vandetanib is about 28:1, about 29:1, about 30:1, about 31:1, about 32:1, about 33:1, about 34:1, about 35:1, about 36:1, about 37:1, or about 38:1. In some embodiments, the molar ratio of carfilzomib to vandetanib is about 33:1.

In some embodiments, the molar ratio of carfilzomib to vandetanib is between about 50:1 to about 200:1 or the molar ratio is any particular value within this range. In some embodiments, the molar ratio of carfilzomib to vandetanib is between about 75:1 to about 175:1. In some embodiments, the molar ratio of carfilzomib to vandetanib is between about 100:1 to about 150:1. In some embodiments, the molar ratio of carfilzomib to vandetanib is between about 115:1 to about 135:1. In some embodiments, the molar ratio of carfilzomib to vandetanib is about 100:1, about 105:1, about 110:1, about 115:1, about 120:1, about 125:1, about 130:1, about 135:1, about 140:1, about 145:1, or about 150:1. In some embodiments, the molar ratio of carfilzomib to vandetanib is about 125:1.

In some embodiments, the molar ratio of carfilzomib to vandetanib is between about 1:1 to about 10:1 or the molar ratio is any particular value within this range. In some embodiments, the molar ratio of carfilzomib to vandetanib is between about 4:1 to about 8:1. In some embodiments, the molar ratio of carfilzomib to vandetanib is between about 5:1 to about 7:1. In some embodiments, the molar ratio of carfilzomib to vandetanib is about 4:1, about 5:1, about 6:1, about 7:1, or about 8:1. In some embodiments, the molar ratio of carfilzomib to vandetanib is about 6:1.

In some embodiments, the carfilzomib is administered in an amount between about 0.1 mg/kg to about 5 mg/kg or at any particular dose within this range. In some embodiments, the carfilzomib is administered in an amount between about 0.1 mg/kg to about 2.5 mg/kg. In some embodiments, the carfilzomib is administered in an amount between about 0.1 mg/kg to about 0.5 mg/kg. In some embodiments, the carfilzomib is administered in an amount between about 0.5 mg/kg to about 1 mg/kg. In some embodiments, the carfilzomib is administered in an amount between about 1 mg/kg to about 1.5 mg/kg. In some embodiments, the carfilzomib is administered in an amount between about 1.5 mg/kg to about 2 mg/kg. In some embodiments, the carfilzomib is administered in an amount between about 2 mg/kg to about 2.5 mg/kg. In some embodiments, the carfilzomib is administered in an amount between about 2.5 mg/kg to about 3 mg/kg. In some embodiments, the carfilzomib is administered in an amount between about 3 mg/kg to about 3.5 mg/kg. In some embodiments, the carfilzomib is administered in an amount between about 3.5 mg/kg to about 4 mg/kg. In some embodiments, the carfilzomib is administered in an amount between about 4 mg/kg to about 4.5 mg/kg. In some embodiments, the carfilzomib is administered in an amount between about 4.5 mg/kg to about 5 mg/kg.

In some embodiments, the vandetanib is administered in an amount between about 0.1 mg/kg to about 25 mg/kg or at any particular dose within this range. In some embodiments, the vandetanib is administered in an amount between about 0.1 mg/kg to about 13 mg/kg. In some embodiments, the vandetanib is administered in an amount between about 0.1 mg/kg to about 8 mg/kg. In some embodiments, the vandetanib is administered in an amount between about 4 mg/kg to about 12 mg/kg. In some embodiments, the vandetanib is administered in an amount between about 8 mg/kg to about 16 mg/kg. In some embodiments, the vandetanib is administered in an amount between about 12 mg/kg to about 20 mg/kg. In some embodiments, the vandetanib is administered in an amount between about 16 mg/kg to about 25 mg/kg. In some embodiments, the vandetanib is administered in an amount between about 0.1 mg/kg to about 3 mg/kg. In some embodiments, the vandetanib is administered in an amount between about 3 mg/kg to about 6 mg/kg. In some embodiments, the vandetanib is administered in an amount between about 6 mg/kg to about 9 mg/kg. In some embodiments, the vandetanib is administered in an amount between about 9 mg/kg to about 12 mg/kg. In some embodiments, the vandetanib is administered in an amount between about 12 mg/kg to about 15 mg/kg. In some embodiments, the vandetanib is administered in an amount between about 15 mg/kg to about 18 mg/kg. In some embodiments, the vandetanib is administered in an amount between about 18 mg/kg to about 21 mg/kg. In some embodiments, the vandetanib is administered in an amount between about 21 mg/kg to about 25 mg/kg.

In some embodiments, the combination of carfilzomib and vandetanib is formulated as a single pharmaceutical composition. Accordingly, in some embodiments, the present disclosure provides a pharmaceutical composition comprising carfilzomib, vandetanib, and a pharmaceutically acceptable carrier. In some embodiments, the combination of carfilzomib and vandetanib is formulated as separate pharmaceutical compositions. Accordingly, in some embodiments, the present disclosure provides (1) a pharmaceutical composition comprising carfilzomib and a pharmaceutically acceptable carrier and (2) a pharmaceutical composition comprising vandetanib and a pharmaceutically acceptable carrier.

The present disclosure also provides a kit comprising the combination of carfilzomib and vandetanib. In some embodiments, the kit comprises instructions for the administration of the combination.

In another aspect, the present disclosure provides a method of treating cancer in a subject in need thereof, including administering to the subject a therapeutically effective amount of carfilzomib and a therapeutically effective amount of vandetanib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene. In some embodiments, the administration of carfilzomib and vandetanib is simultaneous. In some embodiments, the administration of carfilzomib and vandetanib is sequential.

In one aspect, the present disclosure provides a pharmaceutical combination including a therapeutically effective amount of binimetinib and a therapeutically effective amount of ponatinib. In one embodiment, binimetinib and ponatinib are provided in synergistically effective amounts.

In another aspect, the present disclosure provides a method of treating cancer in a subject in need thereof, including administering to the subject a therapeutically effective amount of binimetinib and a therapeutically effective amount of ponatinib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of ponatinib and a therapeutically effective amount of copanlisib. In one embodiment, ponatinib and copanlisib are provided in synergistically effective amounts.

In another aspect, the present disclosure provides a method of treating cancer in a subject in need thereof, including administering to the subject a therapeutically effective amount of ponatinib and a therapeutically effective amount of copanlisib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of avapritinib and a therapeutically effective amount of buparlisib. In one embodiment, avapritinib and buparlisib are provided in synergistically effective amounts.

In another aspect, the present disclosure provides a method of treating cancer in a subject in need thereof, including administering to the subject a therapeutically effective amount of avapritinib and a therapeutically effective amount of buparlisib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of palbociclib and a therapeutically effective amount of sunitinib. In one embodiment, palbociclib and sunitinib are provided in synergistically effective amounts.

In another aspect, the present disclosure provides a method of treating cancer in a subject in need thereof, including administering to the subject a therapeutically effective amount of palbociclib and a therapeutically effective amount of sunitinib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of gilteritinib and a therapeutically effective amount of larotrectinib. In one embodiment, gilteritinib and larotrectinib are provided in synergistically effective amounts.

In another aspect, the present disclosure provides a method of treating cancer in a subject in need thereof, including administering to the subject a therapeutically effective amount of gilteritinib and a therapeutically effective amount of larotrectinib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of copanlisib and a therapeutically effective amount of pazopanib. In one embodiment, copanlisib and pazopanib are provided in synergistically effective amounts.

In another aspect, the present disclosure provides a method of treating cancer in a subject in need thereof, including administering to the subject a therapeutically effective amount of copanlisib and a therapeutically effective amount of pazopanib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of copanlisib and a therapeutically effective amount of pexidartinib. In one embodiment, copanlisib and pexidartinib are provided in synergistically effective amounts.

In another aspect, the present disclosure provides a method of treating cancer in a subject in need thereof, including administering to the subject a therapeutically effective amount of copanlisib and a therapeutically effective amount of pexidartinib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of pazopanib and a therapeutically effective amount of gilteritinib. In one embodiment, pazopanib and gilteritinib are provided in synergistically effective amounts.

In another aspect, the present disclosure provides a method of treating cancer in a subject in need thereof, including administering to the subject a therapeutically effective amount of pazopanib and a therapeutically effective amount of gilteritinib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of binimetinib and a therapeutically effective amount of ibrutinib. In one embodiment, binimetinib and ibrutinib are provided in synergistically effective amounts.

In another aspect, the present disclosure provides a method of treating cancer in a subject in need thereof, including administering to the subject a therapeutically effective amount of binimetinib and a therapeutically effective amount of ibrutinib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of ponatinib and a therapeutically effective amount of ibrutinib. In one embodiment, ponatinib and ibrutinib are provided in synergistically effective amounts.

In another aspect, the present disclosure provides a method of treating cancer in a subject in need thereof, including administering to the subject a therapeutically effective amount of ponatinib and a therapeutically effective amount of ibrutinib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of copanlisib and a therapeutically effective amount of ibrutinib. In one embodiment, copanlisib and ibrutinib are provided in synergistically effective amounts.

In another aspect, the present disclosure provides a method of treating cancer in a subject in need thereof, including administering to the subject a therapeutically effective amount of copanlisib and a therapeutically effective amount of ibrutinib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of ponatinib and a therapeutically effective amount of dasatinib. In one embodiment, ponatinib and dasatinib are provided in synergistically effective amounts.

In another aspect, the present disclosure provides a method of treating cancer in a subject in need thereof, including administering to the subject a therapeutically effective amount of ponatinib and a therapeutically effective amount of dasatinib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of carfilzomib and a therapeutically effective amount of gilteritinib. In one embodiment, carfilzomib and gilteritinib are provided in synergistically effective amounts.

In another aspect, the present disclosure provides a method of treating cancer in a subject in need thereof, including administering to the subject a therapeutically effective amount of carfilzomib and a therapeutically effective amount of gilteritinib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of erlotinib and a therapeutically effective amount of carfilzomib. In one embodiment, erlotinib and carfilzomib are provided in synergistically effective amounts.

In another aspect, the present disclosure provides a method of treating cancer in a subject in need thereof, including administering to the subject a therapeutically effective amount of erlotinib and a therapeutically effective amount of carfilzomib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of carfilzomib and a therapeutically effective amount of ponatinib. In one embodiment, carfilzomib and ponatinib are provided in synergistically effective amounts.

In another aspect, the present disclosure provides a method of treating cancer in a subject in need thereof, including administering to the subject a therapeutically effective amount of carfilzomib and a therapeutically effective amount of ponatinib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of dasatinib and a therapeutically effective amount of carfilzomib. In one embodiment, dasatinib and carfilzomib are provided in synergistically effective amounts.

In another aspect, the present disclosure provides a method of treating cancer in a subject in need thereof, including administering to the subject a therapeutically effective amount of dasatinib and a therapeutically effective amount of carfilzomib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of binimetinib and a therapeutically effective amount of carfilzomib. In one embodiment, binimetinib and carfilzomib are provided in synergistically effective amounts.

In another aspect, the present disclosure provides a method of treating cancer in a subject in need thereof, including administering to the subject a therapeutically effective amount of binimetinib and a therapeutically effective amount of carfilzomib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of carfilzomib and a therapeutically effective amount of ibrutinib. In one embodiment, carfilzomib and ibrutinib are provided in synergistically effective amounts.

In another aspect, the present disclosure provides a method of treating cancer in a subject in need thereof, including administering to the subject a therapeutically effective amount of carfilzomib and a therapeutically effective amount of ibrutinib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of cabozantinib and a therapeutically effective amount of carfilzomib. In one embodiment, cabozantinib and carfilzomib are provided in synergistically effective amounts.

In another aspect, the present disclosure provides a method of treating cancer in a subject in need thereof, including administering to the subject a therapeutically effective amount of cabozantinib and a therapeutically effective amount of carfilzomib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of palbociclib and a therapeutically effective amount of vandetanib. In one embodiment, palbociclib and vandetanib are provided in synergistically effective amounts.

In another aspect, the present disclosure provides a method of treating cancer in a subject in need thereof, including administering to the subject a therapeutically effective amount of palbociclib and a therapeutically effective amount of vandetanib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of vandetanib and a therapeutically effective amount of ponatinib. In one embodiment, vandetanib and ponatinib are provided in synergistically effective amounts.

In another aspect, the present disclosure provides a method of treating cancer in a subject in need thereof, including administering to the subject a therapeutically effective amount of vandetanib and a therapeutically effective amount of ponatinib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of vandetanib and a therapeutically effective amount of copanlisib. In one embodiment, vandetanib and copanlisib are provided in synergistically effective amounts.

In another aspect, the present disclosure provides a method of treating cancer in a subject in need thereof, including administering to the subject a therapeutically effective amount of vandetanib and a therapeutically effective amount of copanlisib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of regorafenib and a therapeutically effective amount of dasatinib. In one embodiment, regorafenib and dasatinib are provided in synergistically effective amounts.

In another aspect, the present disclosure provides a method of treating cancer in a subject in need thereof, including administering to the subject a therapeutically effective amount of regorafenib and a therapeutically effective amount of dasatinib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of regorafenib and a therapeutically effective amount of copanlisib. In one embodiment, regorafenib and copanlisib are provided in synergistically effective amounts.

In another aspect, the present disclosure provides a method of treating cancer in a subject in need thereof, including administering to the subject a therapeutically effective amount of regorafenib and a therapeutically effective amount of copanlisib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of binimetinib and a therapeutically effective amount of dasatinib. In one embodiment, binimetinib and dasatinib are provided in synergistically effective amounts.

In another aspect, the present disclosure provides a method of treating cancer in a subject in need thereof, including administering to the subject a therapeutically effective amount of binimetinib and a therapeutically effective amount of dasatinib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

In one aspect, the present disclosure provides a pharmaceutical combination including a therapeutically effective amount of vandetanib and a therapeutically effective amount of larotrectinib. In one embodiment, vandetanib and larotrectinib are provided in synergistically effective amounts.

In another aspect, the present disclosure provides a method of treating cancer in a subject in need thereof, including administering to the subject a therapeutically effective amount of vandetanib and a therapeutically effective amount of larotrectinib. In one embodiment, the cancer is ovarian cancer. In another embodiment, the cancer is selected from high-grade serous ovarian carcinoma, clear cell ovarian carcinoma, endometrioid ovarian carcinoma, mucinous ovarian carcinoma, and low-grade serous ovarian carcinoma. In another embodiment, the cancer is high-grade serous ovarian cancer. In yet another embodiment, the cancer is high-grade serous ovarian adenocarcinoma. In still another embodiment, the cancer is ovarian cancer characterized by mutation of the TP53 gene.

Delivery forms of the combinations and pharmaceutical compositions of the present disclosure may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. In some embodiments, the compositions are formulated for intravenous infusion, topical administration, or oral administration.

For oral administration, the compounds of the present disclosure can be provided in the form of tablets or capsules, or as a solution, emulsion, or suspension. To prepare the oral compositions, the compounds may be formulated to yield a dosage of, e.g., from about 0.05 to about 100 mg/kg daily, or from about 0.05 to about 35 mg/kg daily, or from about 0.1 to about 10 mg/kg daily. For example, a total daily dosage of about 5 mg to 5 g daily may be accomplished by dosing once, twice, three, or four times per day.

Oral tablets may include one or more compound according to the present disclosure mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are suitable disintegrating agents. Binding agents may include starch and gelatin. A lubricating agent, if present, may be magnesium stearate, stearic acid or talc. If desired, the tablets may be coated with a material such as glyceryl monostearate or glyceryl distearate to delay absorption in the gastrointestinal tract or may be coated with an enteric coating.

Capsules for oral administration include hard and soft gelatin capsules. To prepare hard gelatin capsules, compounds of the present disclosure may be mixed with a solid, semi-solid, or liquid diluent. Soft gelatin capsules may be prepared by mixing the compound of the present disclosure with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this present disclosure may also be administered by non-oral routes. For example, the compositions may be formulated for rectal administration as a suppository. For parenteral use, including intravenous, intramuscular, intraperitoneal, or subcutaneous routes, the compounds of the present disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compounds of the present disclosure may utilize a patch formulation to affect transdermal delivery.

Compounds of the present disclosure may alternatively be administered in methods of this present disclosure by inhalation, via the nasal or oral routes, e.g., in a spray formulation also containing a suitable carrier.

### EXAMPLES

### Example 1. Synergism of Drug Combinations in Human Ovarian Tumor Cell Lines

Various drug combinations were screened in CaOV-3 and OV-90 human ovarian tumor cell lines for synergy using the Bliss independence model. Cells were plated in growth media in 384-well plates in 50 μL volume. Cells were incubated for 24 hours at 37° C. in a humidified incubator prior to application of drug.

| | Growth Media/ | |
|---|---|---|
| Cell Line | Drugging Media | Seeding Density |
| OV-90 | MEM + 15% fetal bovine | 938 cells/well |
| | serum + 1% penicillin- | |
| | streptomycin | |
| CaOV-3 | RPMI-1640 + 10% fetal | 2814 cells/well |
| | bovine serum + 1% | |
| | penicillin-streptomycin | |

Following incubation, 1-2 mg of drug was aliquoted and resuspended in DMSO, using a stock concentration of 10 mM for all drugs except for copanlisib, which was prepared with a stock concentration of 2 mM.

Each plate was serial diluted 1:3 for three replicates per concentration at 10 UM high. For each drug combination, two technical replicates were done. Dplates were incubated for 72 hours at 37° C. in a humidified incubator.

Following incubation, 25 μL of 1:1 mixture of sterile water and CellTiter-Glo® Reagent was added to each well. The plates were incubated for 60 minutes at room temperature. After incubation, luminescence was recorded using a SpectraMax® i3 Plate Reader. Synergy score results were generated using the Bliss Model contained in the SynergyFinder web-based tool to determine the level of combination interaction. The Bliss independent model assumes a stochastic process in which two drugs elicit their effects independently, and the expected combination effect can be calculated based on the probability of independent events:

Bliss predicted inhibition rat (%)=observed inhibition (Drug A alone)+observed inhibition (Drug B alone)−observed inhibtion of combined dose (Drug A+Drug B together)

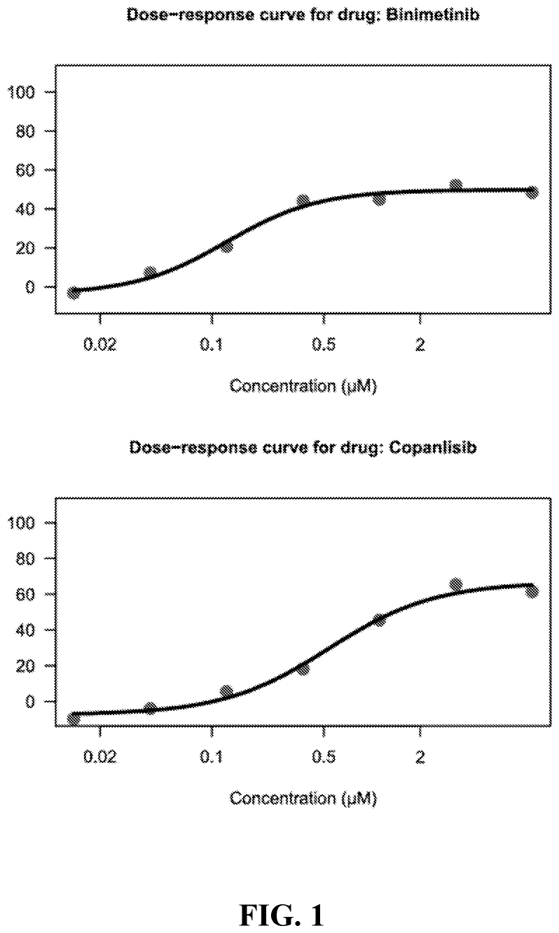

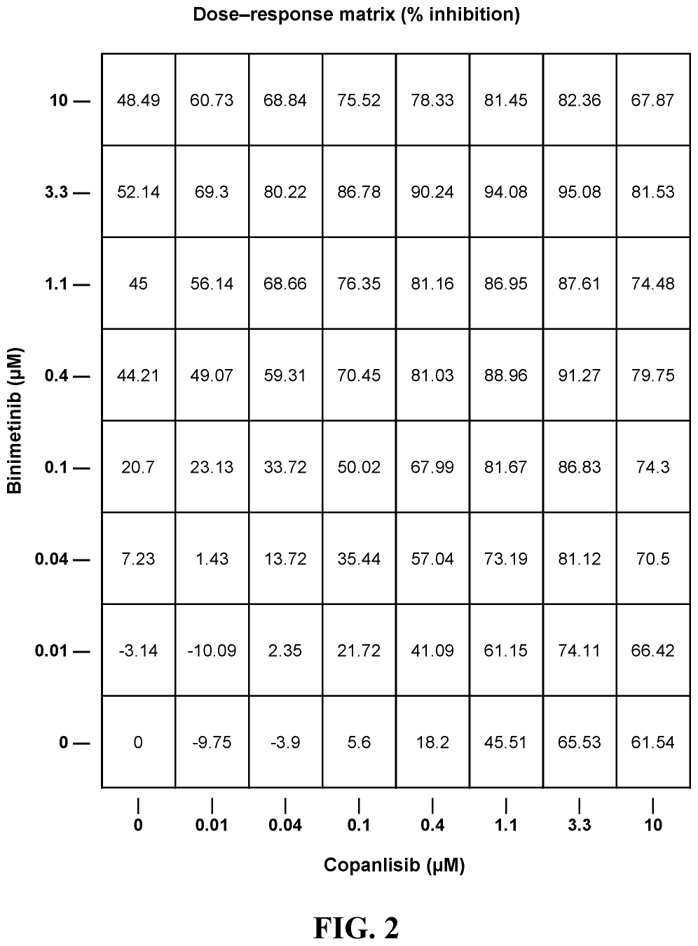

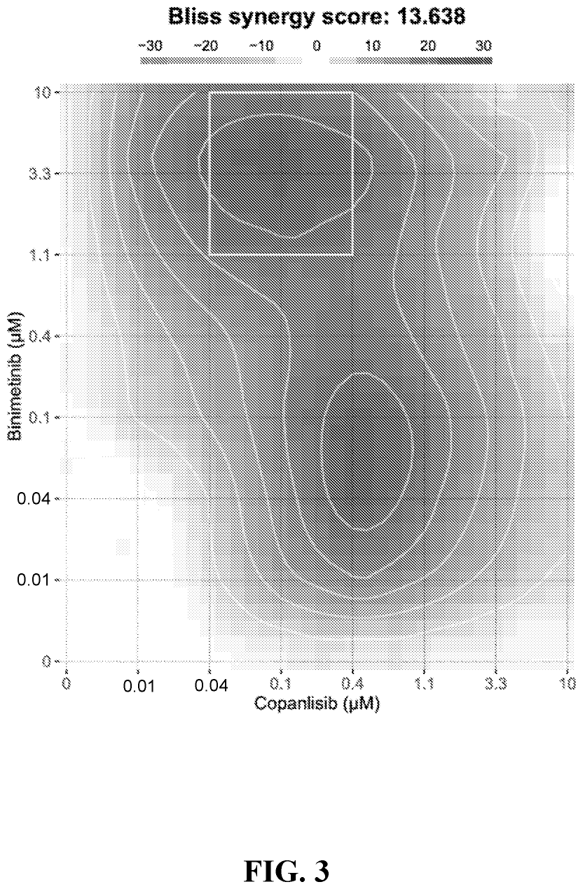

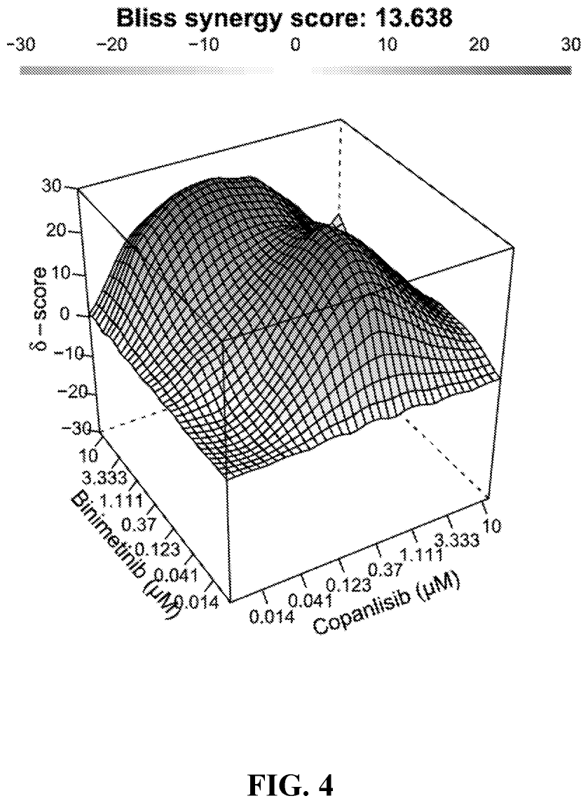

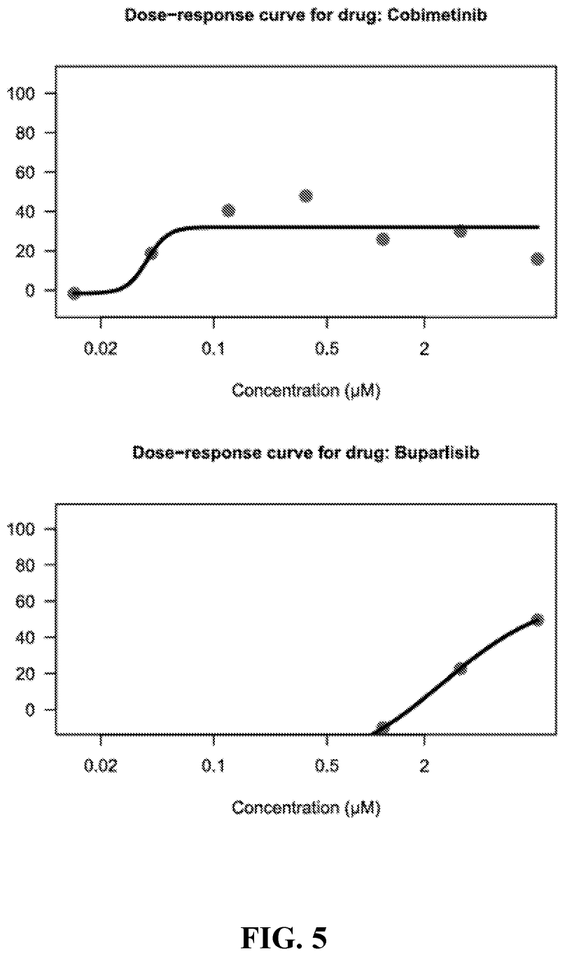

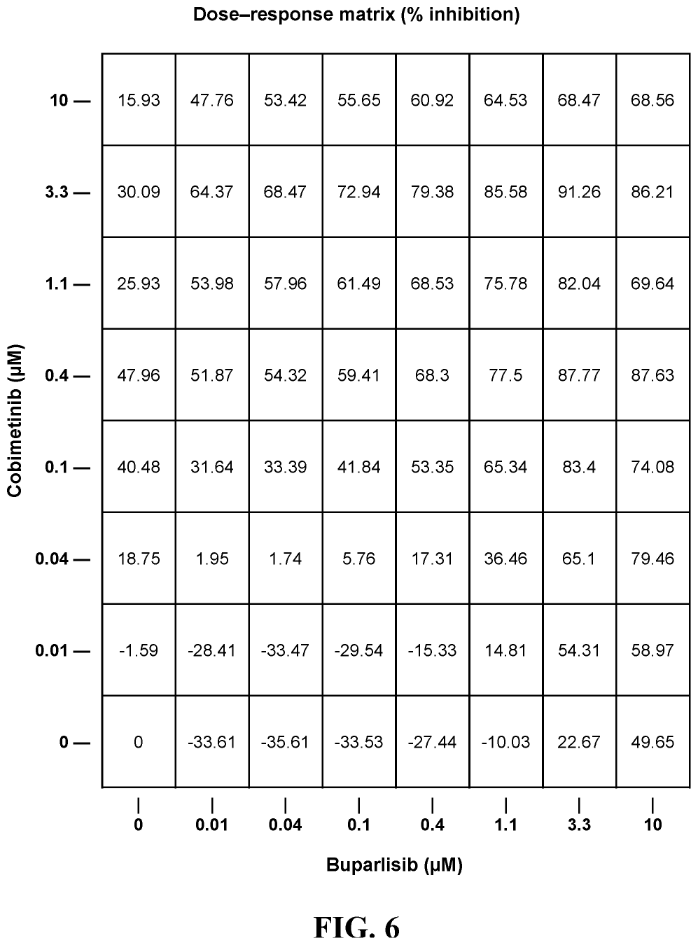

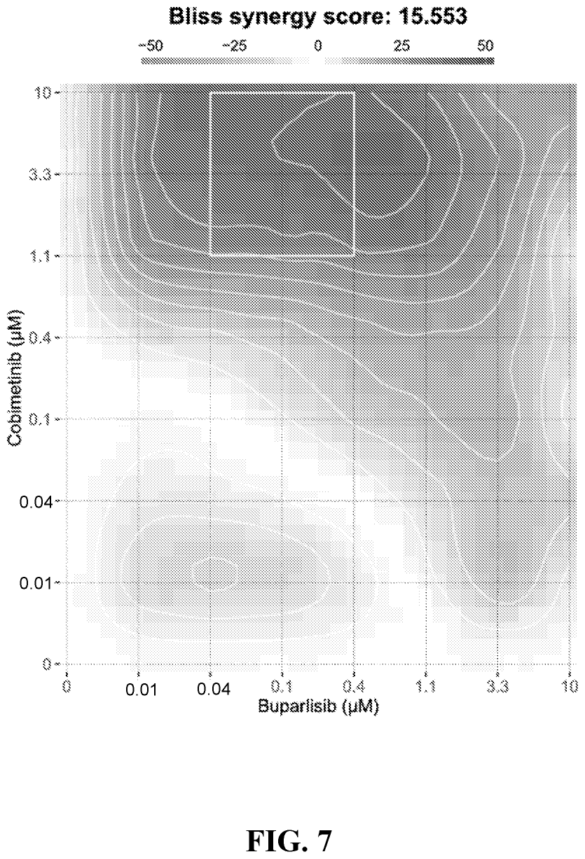

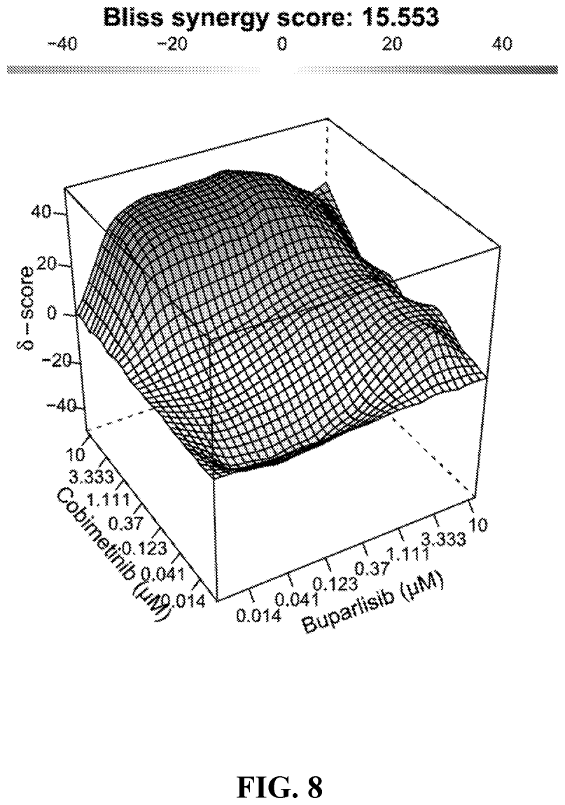

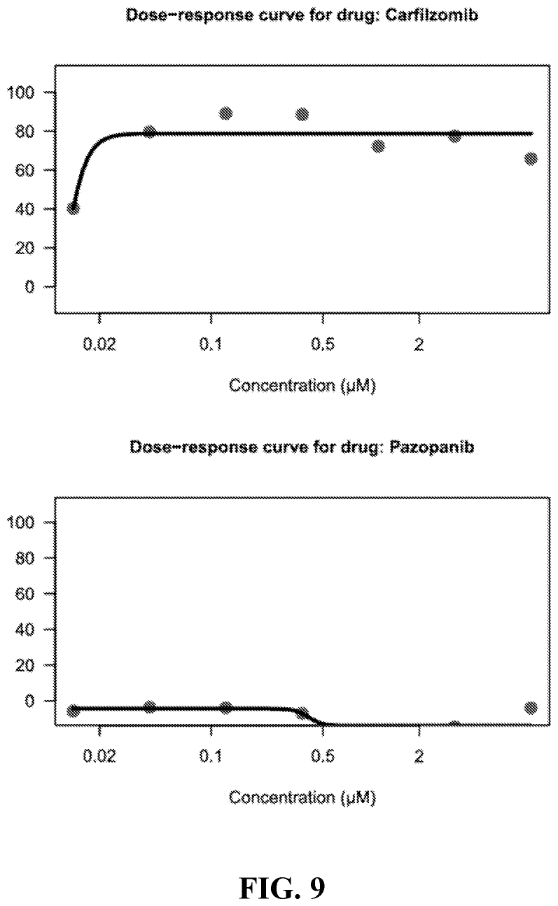

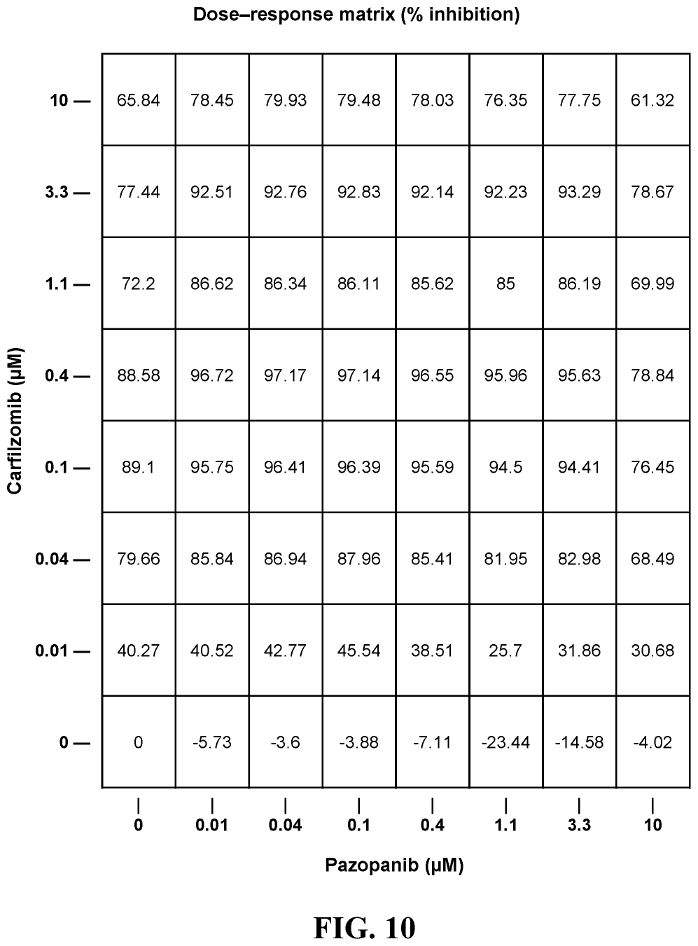

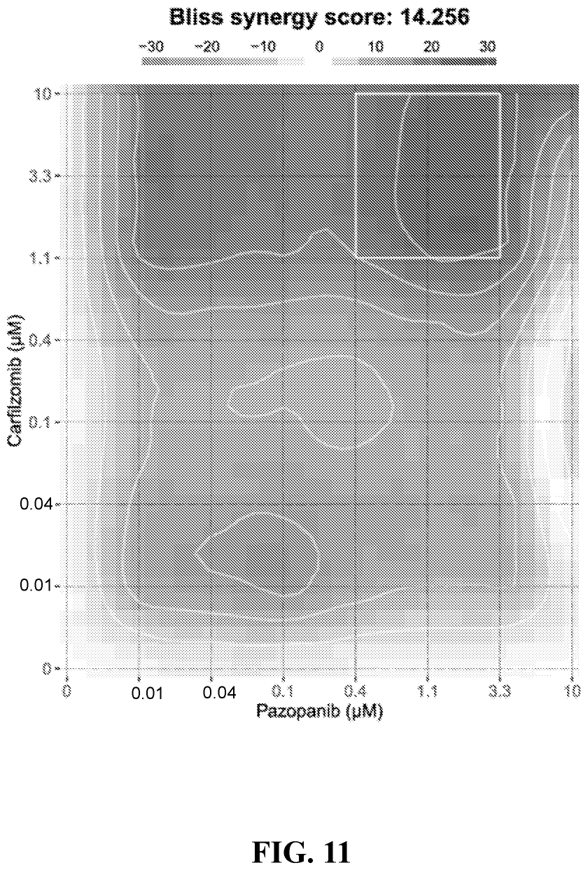

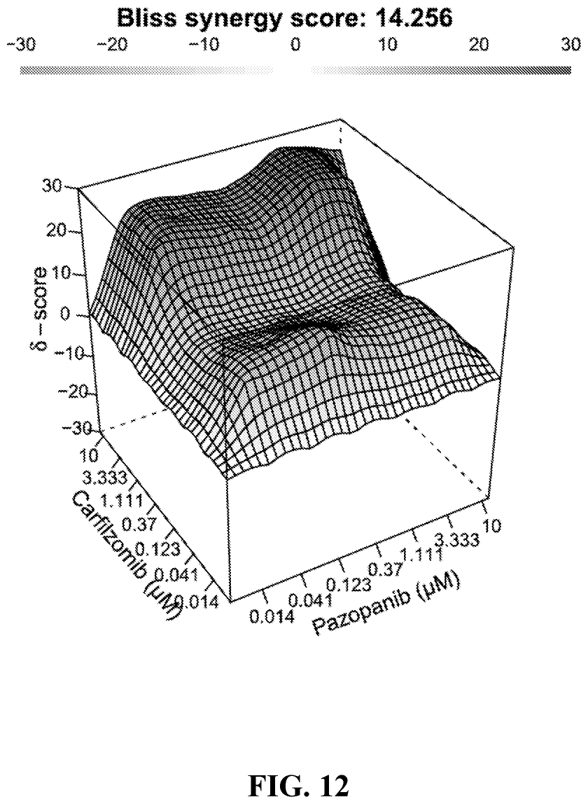

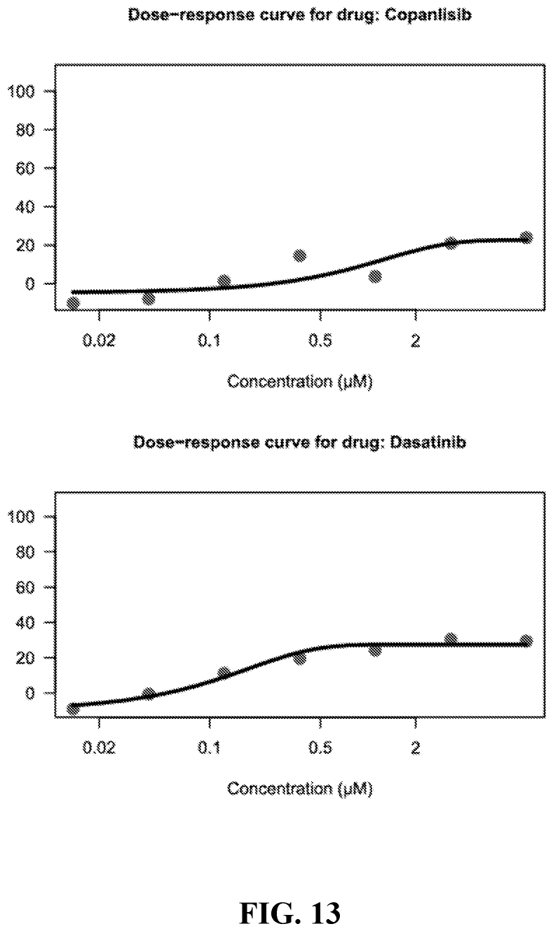

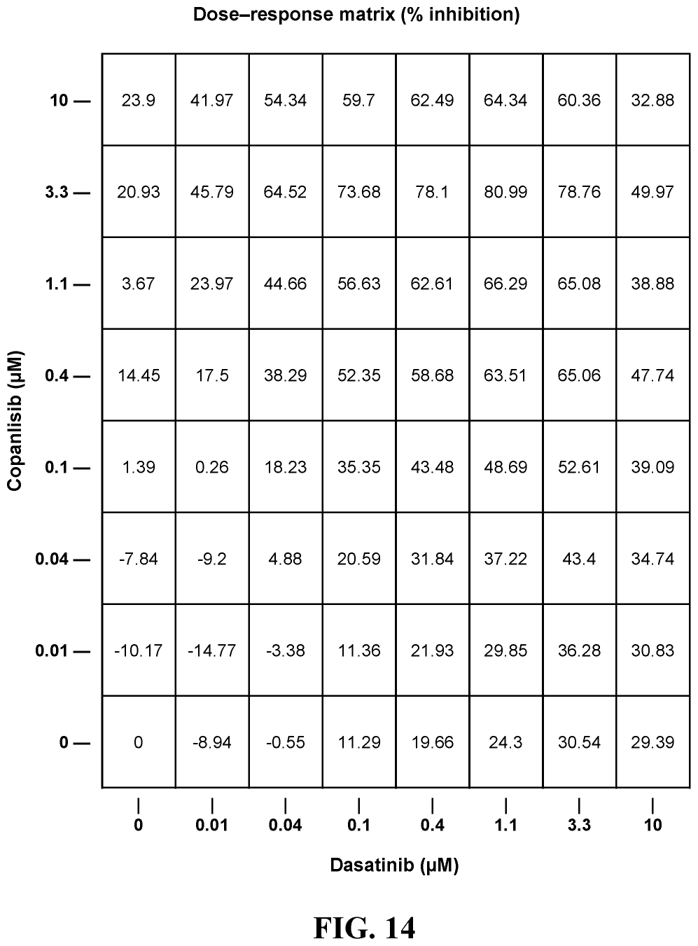

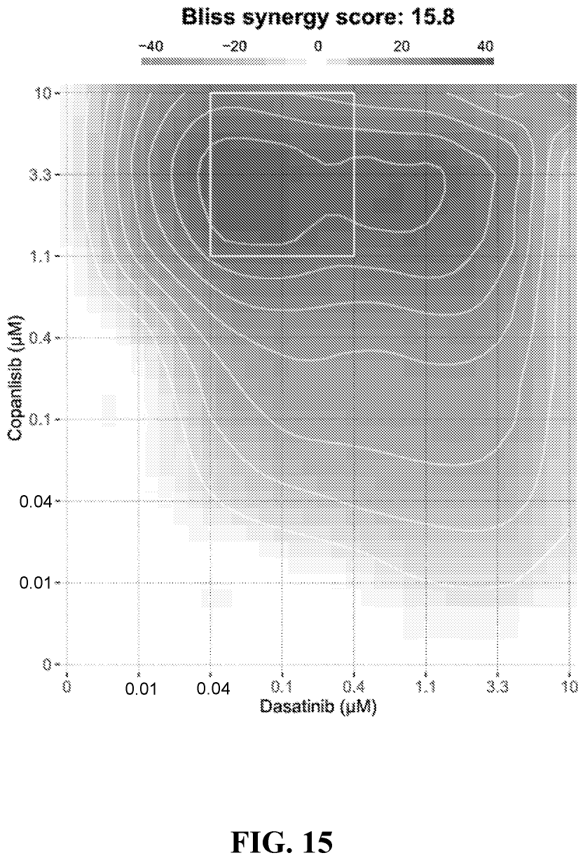

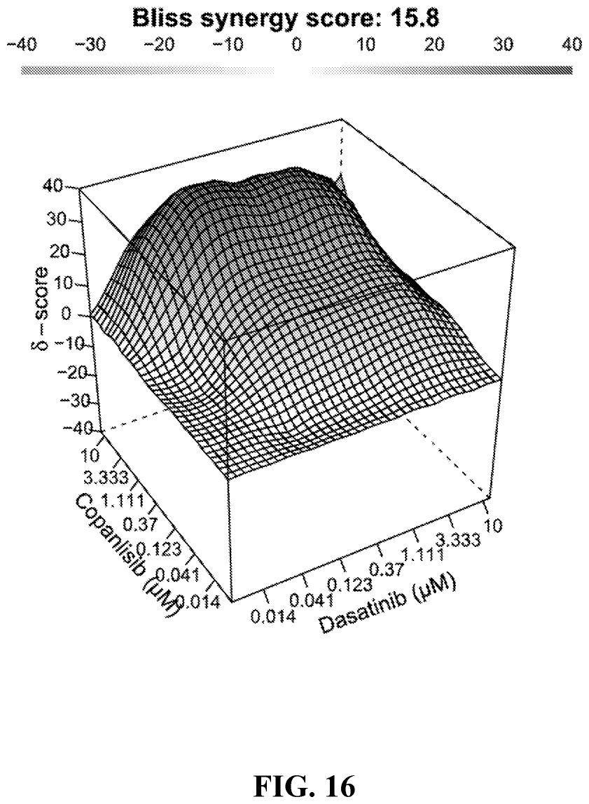

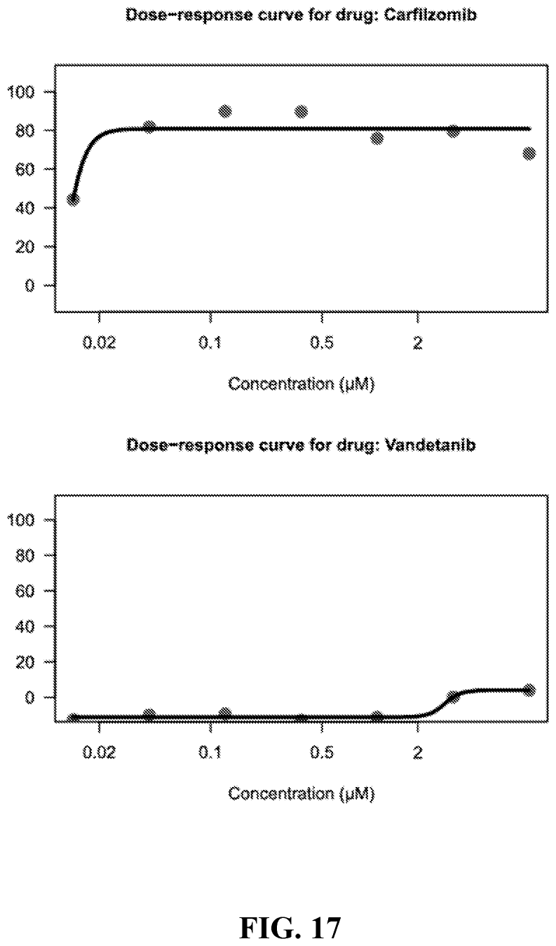

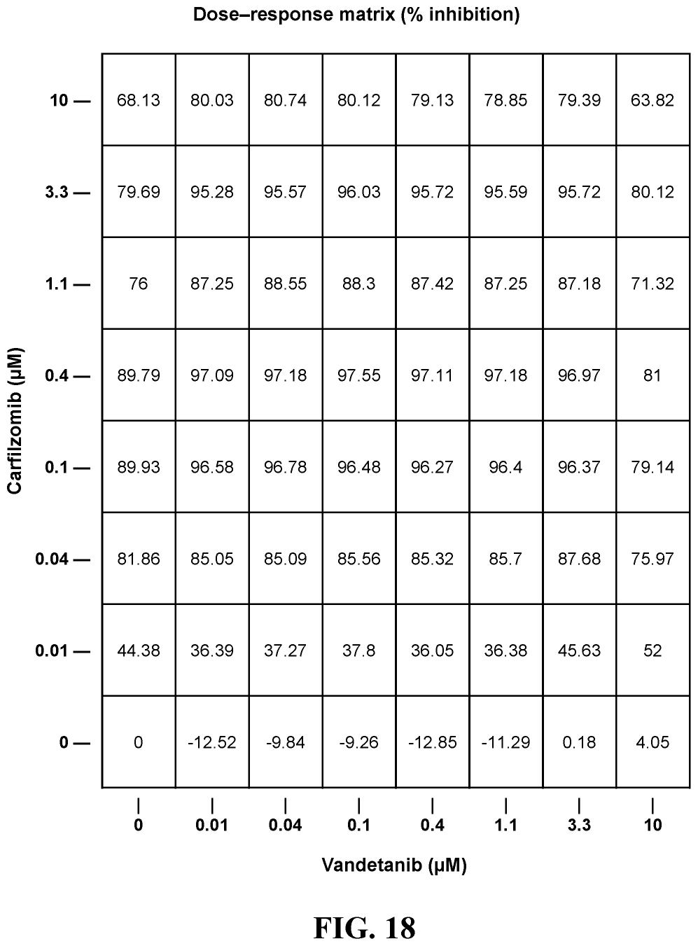

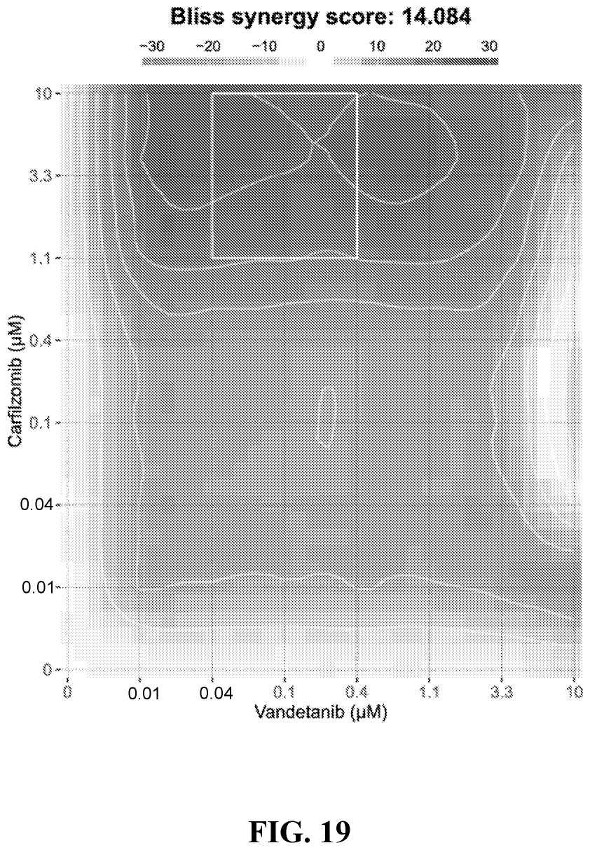

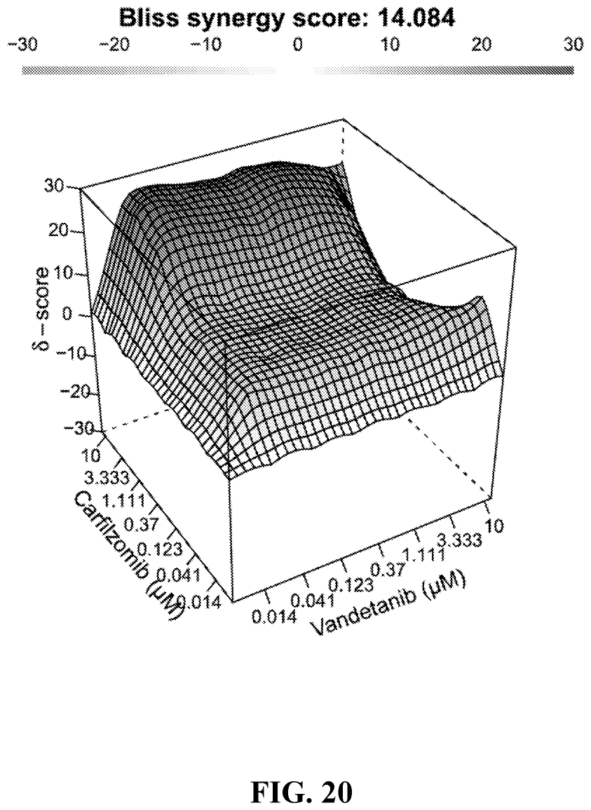

The results, including synergy scores and most synergistic area, are provided in FIGS. 1-20 and in the table below. A Bliss synergy score less than-10 indicates that the interaction between two drugs is likely to be antagonistic. A Bliss synergy score between-10 and 10 indicates that the interaction between two drugs is likely to be additive. A Bliss synergy score above 10 indicates the interaction between two drugs is likely to be synergistic.

| | | | | Max | Most |
|---|---|---|---|---|---|
| | | | Synergy | inhibition | synergistic |
| Cell line | Drug 1 | Drug 2 | score | value (%) | area score |
| OV-90 | Binimetinib | Copanlisib | 13.64 | 95.08 | 23.33 |
| OV-90 | Cobimetinib | Buparlisib | 15.55 | 91.26 | 38.00 |
| OV-90 | Carfilzomib | Pazopanib | 14.26 | 97.17 | 24.72 |
| CaOV-3 | Copanlisib | Dasatinib | 15.80 | 80.99 | 31.06 |
| OV-90 | Carfilzomib | Vandetanib | 14.08 | 97.55 | 24.16 |

The five drug combinations shown in the table above were found to have synergy scores greater than 10. Accordingly, the combinations synergistically inhibit the proliferation of ovarian cancer cells.

### Combinations of Features

Features described above as well as those claimed below may be combined in various ways without departing from the scope hereof. The following examples illustrate possible, non-limiting combinations of features and embodiments described above. It should be clear that other changes and modifications may be made to the present embodiments without departing from the spirit and scope of this invention:
(A1) A combination includes a therapeutically effective amount of binimetinib and a therapeutically effective amount of copanlisib.
(A2) In the combination denoted (A1), the binimetinib and copanlisib are provided in synergistically effective amounts.
(A3) In either of the combinations denoted (A1) and (A2), the molar ratio of binimetinib to copanlisib is between about 23:1 to about 43:1.
(A4) In any of the combinations denoted (A1)-(A3), the molar ratio of binimetinib to copanlisib is between about 28:1 to about 38:1.
(A5) In any of the combinations denoted (A1)-(A4), the molar ratio of binimetinib to copanlisib is between about 33.1.
(A6) In either of the combinations denoted (A1) and (A2), the molar ratio of binimetinib to copanlisib is between about 1:4 to about 1:10.
(A7) In any of the combinations denoted (A1), (A2), and (A6), the molar ratio of binimetinib to copanlisib is between about 1:6 to about 1:8.
(A8) In any of the combinations denoted (A1), (A2), (A6), and (A7), the molar ratio of binimetinib to copanlisib is about 1:7.
(B1) A method of treating cancer in a subject in need thereof comprises administering to the subject a therapeutically effective amount of any of the combinations denoted (A1)-(A8).
(B2) In the method denoted (B1), the cancer is ovarian cancer.
(B3) In the method denoted (B1), the ovarian cancer is selected from high-grade serous ovarian carcinoma, clear cell ovarian carcinoma, endometrioid ovarian carcinoma, mucinous ovarian carcinoma, and low-grade serous ovarian carcinoma.
(B4) In the method denoted (B1), the ovarian cancer is high-grade serous ovarian cancer.
(B5) In the method denoted (B1), the ovarian cancer is high-grade serous ovarian adenocarcinoma.
(B6) In the method denoted (B1), the ovarian cancer is characterized by mutation of the TP53 gene.
(B7) In any of the methods denoted (B1)-(B6), the administration of binimetinib and copanlisib is simultaneous.
(B8) In any of the methods denoted (B1)-(B6), the administration of binimetinib and copanlisib is sequential.
(C1) A combination comprises a therapeutically effective amount of cobimetinib and a therapeutically effective amount of buparlisib.
(C2) In the combination of (C1), the cobimetinib and buparlisib are provided in synergistically effective amounts.
(C3) In either of the combinations denoted (C1) and (C2), the molar ratio of cobimetinib to buparlisib is between about 23:1 to about 43:1.
(C4) In any of the combinations denoted (C1)-(C3), the molar ratio of cobimetinib to buparlisib is between about 28:1 to about 38:1.
(C5) In any of the combinations denoted (C1)-(C4), the molar ratio of cobimetinib to buparlisib is about 33:1.
(C6) In either of the combinations denoted (C1) and (C2), the molar ratio of cobimetinib to buparlisib is between about 5:1 to about 15:1.
(C7) In any of the combinations denoted (C1), (C2), and (C6), the molar ratio of cobimetinib to buparlisib is between about 8:1 to about 12:1.
(C8) In any of the combinations denoted (C1), (C2), (C6), and (C7), the molar ratio of cobimetinib to buparlisib is about 10:1.
(D1) A method of treating cancer in a subject in need thereof comprises administering to the subject a therapeutically effective amount of any of the combinations denoted (C1)-(C8).
(D2) In the method denoted (D1), the cancer is ovarian cancer.
(D3) In the method denoted (D1), the ovarian cancer is selected from high-grade serous ovarian carcinoma, clear cell ovarian carcinoma, endometrioid ovarian carcinoma, mucinous ovarian carcinoma, and low-grade serous ovarian carcinoma.
(D4) In the method denoted (D1), the ovarian cancer is high-grade serous ovarian cancer.
(D5) In the method denoted (D1), the ovarian cancer is high-grade serous ovarian adenocarcinoma.
(D6) In the method denoted (D1), the ovarian cancer is characterized by mutation of the TP53 gene.
(D7) In any of the methods denoted (D1)-(D6), the administration of cobimetinib and buparlisib is simultaneous.
(D8) In any of the methods denoted (D1)-(D6), the administration of cobimetinib and buparlisib is sequential.
(E1) A combination comprises a therapeutically effective amount of carfilzomib and a therapeutically effective amount of pazopanib.
(E2) In the combination denoted (E1), the carfilzomib and pazopanib are provided in synergistically effective amounts.
(E3) In either of the combinations denoted (E1) and (E2), the molar ratio of carfilzomib to pazopanib is between about 1:1 to about 6:1.
(E4) In any of the combinations denoted (E1)-(E3), the molar ratio of carfilzomib to pazopanib is between about 2:1 to about 5:1.
(E5) In any of the combinations denoted (E1)-(E4), the molar ratio of carfilzomib to pazopanib is about 3:1.
(E6) In any of the combinations denoted (E1)-(E4), the molar ratio of carfilzomib to pazopanib is about 4:1.
(F1) A method of treating cancer in a subject in need thereof comprises administering to the subject a therapeutically effective amount of any of the combinations denoted (E1)-(E6).
(F2) In the method denoted (F1), the cancer is ovarian cancer.
(F3) In the method denoted (F1), the ovarian cancer is selected from high-grade serous ovarian carcinoma, clear cell ovarian carcinoma, endometrioid ovarian carcinoma, mucinous ovarian carcinoma, and low-grade serous ovarian carcinoma.
(F4) In the method denoted (F1), the ovarian cancer is high-grade serous ovarian cancer.
(F5) In the method denoted (F1), the ovarian cancer is high-grade serous ovarian adenocarcinoma.
(F6) In the method denoted (F1), the ovarian cancer is characterized by mutation of the TP53 gene.
(F7) In any of the methods denoted (F1)-(F6), the administration of carfilzomib and pazopanib is simultaneous.
(F8) In any of the methods denoted (F1)-(F6), the administration of carfilzomib and pazopanib is sequential.
(G1) A combination comprises a therapeutically effective amount of copanlisib and a therapeutically effective amount of dasatinib.
(G2) In the combination denoted (G1), the copanlisib and dasatinib are provided in synergistically effective amounts.
(G3) In either of the combinations denoted (G1) and (G2), the molar ratio of copanlisib to dasatinib is between about 23:1 to about 43:1.
(G4) In any of the combinations denoted (G1)-(G3), the molar ratio of copanlisib to dasatinib is between about 28:1 to about 38:1.
(G5) In any of the combinations denoted (G1)-(G4), the molar ratio of copanlisib to dasatinib is about 33:1.
(H1) A method of treating cancer in a subject in need thereof comprises administering to the subject a therapeutically effective amount of any of the combinations denoted (G1)-(G5).
(H2) In the method denoted (H1), the cancer is ovarian cancer.
(H3) In the method denoted (H1), the ovarian cancer is selected from high-grade serous ovarian carcinoma, clear cell ovarian carcinoma, endometrioid ovarian carcinoma, mucinous ovarian carcinoma, and low-grade serous ovarian carcinoma.
(H4) In the method denoted (H1), the ovarian cancer is high-grade serous ovarian cancer.
(H5) In the method denoted (H1), the ovarian cancer is high-grade serous ovarian adenocarcinoma.
(H6) In the method denoted (H1), the ovarian cancer is characterized by mutation of the TP53 gene.
(H7) In any of the methods denoted (H1)-(H6), the administration of copanlisib and dasatinib is simultaneous.
(H8) In any of the methods denoted (H1)-(H6), the administration of copanlisib and dasatinib is sequential.
(11) A combination comprises a therapeutically effective amount of carfilzomib and a therapeutically effective amount of vandetanib.
(12) In the combination denoted (11), the carfilzomib and vandetanib are provided in synergistically effective amounts.
(13) In either of the combinations denoted (11) and (12), the molar ratio of carfilzomib to vandetanib is between about 23:1 to about 43:1.
(14) In any of the combinations denoted (11)-(13), the molar ratio of carfilzomib to vandetanib is between about 28:1 to about 38:1.
(15) In any of the combinations denoted (11)-(14), the molar ratio of carfilzomib to vandetanib is about 33:1.
(16) In either of the combinations denoted (11) and (12), the molar ratio of carfilzomib to vandetanib is between about 75:1 to about 175:1.
(17) In any of the combinations denoted (11), (12), and (16), the molar ratio of carfilzomib to vandetanib is between about 100:1 to about 150:1.
(18) In any of the combinations denoted (11), (12), (16), and (17), the molar ratio of carfilzomib to vandetanib is about 125:1.
(19) In either of the combinations denoted (11) and (12), the molar ratio of carfilzomib to vandetanib is between about 4:1 to about 8:1.
(110) In any of the combinations denoted (11), (12), and (19), the molar ratio of carfilzomib to vandetanib is between about 5:1 to about 7:1.
(111) In any of the combinations denoted (11), (12), (19), and (110), the molar ratio of carfilzomib to vandetanib is about 6:1.
(J1) A method of treating cancer in a subject in need thereof comprises administering to the subject a therapeutically effective amount of any of the combinations denoted (11)-(111).
(J2) In the method denoted (J1), the cancer is ovarian cancer.
(J3) In the method denoted (J1), the ovarian cancer is selected from high-grade serous ovarian carcinoma, clear cell ovarian carcinoma, endometrioid ovarian carcinoma, mucinous ovarian carcinoma, and low-grade serous ovarian carcinoma.
(J4) In the method denoted (J1), the ovarian cancer is high-grade serous ovarian cancer.
(J5) In the method denoted (J1), the ovarian cancer is high-grade serous ovarian adenocarcinoma.
(J6) In the method denoted (J1), the ovarian cancer is characterized by mutation of the TP53 gene.
(J7) In any of the methods denoted (J1)-(J6), the administration of carfilzomib and vandetanib is simultaneous.
(J8) In any of the methods denoted (J1)-(J6), the administration of carfilzomib and vandetanib is sequential.

What is claimed is:

1. A combination comprising a therapeutically effective amount of binimetinib and a therapeutically effective amount of copanlisib.

2. The combination of claim 1, wherein the binimetinib and copanlisib are provided in synergistically effective amounts.

3. The combination of claim 1, wherein the molar ratio of binimetinib to copanlisib is between about 23:1 to about 43:1.

4. The combination of claim 3, wherein the molar ratio of binimetinib to copanlisib is between about 28:1 to about 38:1.

5. The combination of claim 4, wherein the molar ratio of binimetinib to copanlisib is about 33:1.

6. The combination of claim 1, wherein the molar ratio of binimetinib to copanlisib is between about 1:4 to about 1:10.

7. The combination of claim 6, wherein the molar ratio of binimetinib to copanlisib is between about 1:6 to about 1:8.

8. The combination of claim 7, wherein the molar ratio of binimetinib to copanlisib is about 1:7.

9. A pharmaceutical composition comprising the combination of any one of claims 1-8 and a pharmaceutically acceptable carrier.

10. A method of treating cancer in a subject in need thereof, comprising administering to the subject a therapeutically effective amount of the combination of any one of claims 1-8 or the pharmaceutical composition of claim 9.

11. The method of claim 10, wherein the cancer is ovarian cancer.

12. The method of claim 11, wherein the ovarian cancer is selected from high-grade serous ovarian carcinoma, clear cell ovarian carcinoma, endometrioid ovarian carcinoma, mucinous ovarian carcinoma, and low-grade serous ovarian carcinoma.

13. The method of claim 11, wherein the ovarian cancer is high-grade serous ovarian cancer.

14. The method of claim 11, wherein the ovarian cancer is high-grade serous ovarian adenocarcinoma.

15. The method of claim 11, wherein the ovarian cancer is characterized by mutation of the TP53 gene.

16. The method of any one of claims 10-15, wherein the administration of binimetinib and copanlisib is simultaneous.

17. The method of any one of claims 10-15, wherein the administration of binimetinib and copanlisib is sequential.

18. A combination comprising a therapeutically effective amount of cobimetinib and a therapeutically effective amount of buparlisib.

19. The combination of claim 18, wherein the cobimetinib and buparlisib are provided in synergistically effective amounts.

20. The combination of claim 18, wherein the molar ratio of cobimetinib to buparlisib is between about 23:1 to about 43:1.

21. The combination of claim 20, wherein the molar ratio of cobimetinib to buparlisib is between about 28:1 to about 38:1.

22. The combination of claim 21, wherein the molar ratio of cobimetinib to buparlisib is about 33:1.

23. The combination of claim 18, wherein the molar ratio of cobimetinib to buparlisib is between about 5:1 to about 15:1.

24. The combination of claim 23, wherein the molar ratio of cobimetinib to buparlisib is between about 8:1 to about 12:1.

25. The combination of claim 24, wherein the molar ratio of cobimetinib to buparlisib is about 10:1.

26. A pharmaceutical composition comprising the combination of any one of claims 18-25 and a pharmaceutically acceptable carrier.

27. A method of treating cancer in a subject in need thereof, comprising administering to the subject a therapeutically effective amount of the combination of any one of claims 17-24 or the pharmaceutical composition of claim 26.

28. The method of claim 27, wherein the cancer is ovarian cancer.

29. The method of claim 28, wherein the ovarian cancer is selected from high-grade serous ovarian carcinoma, clear cell ovarian carcinoma, endometrioid ovarian carcinoma, mucinous ovarian carcinoma, and low-grade serous ovarian carcinoma.

30. The method of claim 28, wherein the ovarian cancer is high-grade serous ovarian cancer.

31. The method of claim 28, wherein the ovarian cancer is high-grade serous ovarian adenocarcinoma.

32. The method of claim 28, wherein the ovarian cancer is characterized by mutation of the TP53 gene.

33. The method of any one of claims 27-32, wherein the administration of cobimetinib and buparlisib is simultaneous.

34. The method of any one of claims 27-32, wherein the administration of cobimetinib and buparlisib is sequential.

35. A combination comprising a therapeutically effective amount of carfilzomib and a therapeutically effective amount of pazopanib.

36. The combination of claim 35, wherein the carfilzomib and pazopanib are provided in synergistically effective amounts.

37. The combination of claim 35, wherein the molar ratio of carfilzomib to pazopanib is between about 1:1 to about 6:1.

38. The combination of claim 37, wherein the molar ratio of carfilzomib to pazopanib is between about 2:1 to about 5:1.

39. The combination of claim 38, wherein the molar ratio of carfilzomib to pazopanib is about 3:1.

40. The combination of claim 36, wherein the molar ratio of carfilzomib to pazopanib is about 4:1.

41. A pharmaceutical composition comprising the combination of any one of claims 35-40 and a pharmaceutically acceptable carrier.

42. A method of treating cancer in a subject in need thereof, comprising administering to the subject a therapeutically effective amount of the combination of any one of claims 35-40 or the pharmaceutical composition of claim 41.

43. The method of claim 42, wherein the cancer is ovarian cancer.

44. The method of claim 43, wherein the ovarian cancer is selected from high-grade serous ovarian carcinoma, clear cell ovarian carcinoma, endometrioid ovarian carcinoma, mucinous ovarian carcinoma, and low-grade serous ovarian carcinoma.

45. The method of claim 43, wherein the ovarian cancer is high-grade serous ovarian cancer.

46. The method of claim 43, wherein the ovarian cancer is high-grade serous ovarian adenocarcinoma.

47. The method of claim 43, wherein the ovarian cancer is characterized by mutation of the TP53 gene.

48. The method of any one of claims 42-47, wherein the administration of carfilzomib and pazopanib is simultaneous.

49. The method of any one of claims 42-47, wherein the administration of carfilzomib and pazopanib is sequential.

50. A combination comprising a therapeutically effective amount of copanlisib and a therapeutically effective amount of dasatinib.

51. The combination of claim 50, wherein the copanlisib and dasatinib are provided in synergistically effective amounts.

52. The combination of claim 50, wherein the molar ratio of copanlisib to dasatinib is between about 23:1 to about 43:1.

53. The combination of claim 52, wherein the molar ratio of copanlisib to dasatinib is between about 28:1 to about 38:1.

54. The combination of claim 53, wherein the molar ratio of copanlisib to dasatinib is about 33:1.

55. A pharmaceutical composition comprising the combination of any one of claims 50-54 and a pharmaceutically acceptable carrier.

56. A method of treating cancer in a subject in need thereof, comprising administering to the subject a therapeutically effective amount of the combination of any one of claims 50-54 or the pharmaceutical composition of claim 55.

57. The method of claim 56, wherein the cancer is ovarian cancer.

58. The method of claim 57, wherein the ovarian cancer is selected from high-grade serous ovarian carcinoma, clear cell ovarian carcinoma, endometrioid ovarian carcinoma, mucinous ovarian carcinoma, and low-grade serous ovarian carcinoma.

59. The method of claim 57, wherein the ovarian cancer is high-grade serous ovarian cancer.

60. The method of claim 57, wherein the ovarian cancer is high-grade serous ovarian adenocarcinoma.

61. The method of claim 57, wherein the ovarian cancer is characterized by mutation of the TP53 gene.

62. The method of any one of claims 56-61, wherein the administration of copanlisib and dasatinib is simultaneous.

63. The method of any one of claims 56-61, wherein the administration of copanlisib and dasatinib is sequential.

64. A combination comprising a therapeutically effective amount of carfilzomib and a therapeutically effective amount of vandetanib.

65. The combination of claim 64, wherein the carfilzomib and vandetanib are provided in synergistically effective amounts.

66. The combination of claim 64, wherein the molar ratio of carfilzomib to vandetanib is between about 23:1 to about 43:1.

67. The combination of claim 66, wherein the molar ratio of carfilzomib to vandetanib is between about 28:1 to about 38:1.

68. The combination of claim 67, wherein the molar ratio of carfilzomib to vandetanib is about 33:1.

69. The combination of claim 64, wherein the molar ratio of carfilzomib to vandetanib is between about 75:1 to about 175:1.

70. The combination of claim 69, wherein the molar ratio of carfilzomib to vandetanib is between about 100:1 to about 150:1.

71. The combination of claim 70, wherein the molar ratio of carfilzomib to vandetanib is about 125:1.

72. The combination of claim 64, wherein the molar ratio of carfilzomib to vandetanib is between about 4:1 to about 8:1.

73. The combination of claim 72, wherein the molar ratio of carfilzomib to vandetanib is between about 5:1 to about 7:1.

74. The combination of claim 73, wherein the molar ratio of carfilzomib to vandetanib is about 6:1.

75. A pharmaceutical composition comprising the combination of any one of claims 64-74 and a pharmaceutically acceptable carrier.

76. A method of treating cancer in a subject in need thereof, comprising administering to the subject a therapeutically effective amount of the combination of any one of claims 64-74 or the pharmaceutical composition of claim 75.

77. The method of claim 76, wherein the cancer is ovarian cancer.

78. The method of claim 77, wherein the ovarian cancer is selected from high-grade serous ovarian carcinoma, clear cell ovarian carcinoma, endometrioid ovarian carcinoma, mucinous ovarian carcinoma, and low-grade serous ovarian carcinoma.

79. The method of claim 77, wherein the ovarian cancer is high-grade serous ovarian cancer.

80. The method of claim 77, wherein the ovarian cancer is high-grade serous ovarian adenocarcinoma.

81. The method of claim 77, wherein the ovarian cancer is characterized by mutation of the TP53 gene.

82. The method of any one of claims 76-81, wherein the administration of carfilzomib and vandetanib is simultaneous.

83. The method of any one of claims 76-81, wherein the administration of carfilzomib and vandetanib is sequential.
